(19) United States
(12) Patent Application Publication
(10) Pub. No.: US 2023/0108258 A1
(43) Pub. Date: Apr. 6, 2023

(54) ANTIBODY-FREE RAPID DETECTION OF SARS-COV-2 PROTEINS USING CORONA PHASE MOLECULAR RECOGNITION (COPHMORE)

(71) Applicant: Massachusetts Institute of Technology, Cambridge, MA (US)

(72) Inventors: Sooyeon Cho, Boston, MA (US); Michael Strano, Lexington, MA (US); Xiaojia Jin, Cambridge, MA (US); Xun Gong, Boston, MA (US); Sungyun Yang, Cambridge, MA (US); Jianqiao Cui, Cambridge, MA (US)

(73) Assignee: Massachusetts Institute of Technology, Cambridge, MA (US)

(21) Appl. No.: 17/881,754

(22) Filed: Aug. 5, 2022

Related U.S. Application Data
(60) Provisional application No. 63/230,178, filed on Aug. 6, 2021.

(51) Int. Cl. C12Q 1/70; C08G 65/333; B82Y 30/00

(52) U.S. Cl. CPC C12Q 1/70; C08G 65/33396; B82Y 30/00

(57) ABSTRACT

Corona phase molecular recognition (CoPhMoRe) that enables the molecular recognition of SARS-CoV-2 viral proteins without the need for antibody or enzymatic receptor incorporation.

### CLAIM OF PRIORITY

This application claims priority to U.S. Provisional Patent Application No. 63/230,178, filed Aug. 6, 2021, which is incorporated by reference in its entirety.

### STATEMENT REGARDING FEDERALLY SPONSORED RESEARCH OR DEVELOPMENT

This invention was made with Government support under Grant No. R42 DE030829 awarded by the National Institutes of Health. The Government has certain rights in the invention.

### FIELD OF THE INVENTION

This invention relates to sensors and methods of detecting viruses, particularly coronavirus.

### BACKGROUND

Rapid and accurate detection of severe acute respiratory syndrome coronavirus 2 (SARS-CoV-2) infection is critical for reducing morbidity of Coronavirus Disease 2019 (COVID-19) (ref. 1). The current methodology in assessing the present infection of SARS-CoV-2 relies on two analytics: (i) nucleic acid-based tests (NAT) which detect the genetic material (RNA) from SARS-CoV-2 and (ii) serological tests which detect the presence of antibodies (IgG and IgM) against SARS-CoV-2 (ref. 2). NAT primarily uses reverse-transcription polymerase chain reaction (RT-PCR) to amplify and detect the SARS-CoV-2 RNA in the patient. Since RT-PCR demonstrate superior sensitivity for viral RNA detection with a limit of detection (LOD) of a few viral RNA copies, the NAT is the preferred testing methods to diagnose SARS-CoV-2 positive cases to date (ref. 3). However, this method is composed of highly complex processes which requires specialized equipment, trained personnel, and long turnaround time limiting the high-throughput diagnosis of large populations and wide testing accessibility in rural regions (refs. 4-6). A serological test, in general, is based on lateral flow immunoassay (LFA) platform, which is relatively easy, inexpensive having a short turnaround time, and amenable to point-of-care diagnostic methodologies (ref. 7). However, the sensitivity of antigen test is generally not high enough to accurately screen the positive cases and provide past viral infections data. Thus, it is hard to identify current active cases of SARS-CoV-2 infection (refs. 7-9). Therefore, there has been a strong drive to find next-generation viral testing technology for faster and simpler diagnostics of large populations, which can directly detect viral antigens in clinical samples without complicated sample preparation steps. In addition, even though NAT and LFA based diagnostics have a lot of advantages, wide deployment of these test kits was not possible until after few months of early phase of pandemic due to complicated development process including sensor design, validation, and implementation, and shortages of laboratory item (refs. 10-11). Thus, accelerated point-of-care sensor development process should be developed for future viral targets.

Several potentially useful new analytics for rapid and sensitive SARS-CoV-2 detection have emerged for diagnosis of COVID-19 against RT-PCR and serological test, including field-effect transistor (FET) sensing devices (refs. 12-14), electrochemical cell devices (refs. 15-17), plasmonic resonance platform (refs. 18-20), optical nanosensors (refs. 21-23), and chemiresistor (ref. 24). These analytic tools showed sensitive and rapid detection performances based on simple sensor signal readout without complex sample processing and specialized equipment. However, all of these methods are based on specific and complex surface chemistry design of substrate and transducers using subunit protein antibody and protein receptor such as DNA/RNA aptamers and angiotensin-converting enzyme 2 (ACE2) to selectively capture the SARS-CoV-2 viral proteins (refs. 25-27). These antibody or protein receptor functionalizations are expensive, fragile, prone to loss of biological activity with external treatment such as immobilization and device interfacing, and exhibit significant batch-dependent variations (refs. 28-29). In addition, they involve multiple processing step of fabrications including incubation, protein synthesis, and washing steps that require a significant amount of time to perform, limiting their use in widespread applications (ref. 30).

### SUMMARY

This Summary introduces a selection of concepts in simplified form that are described further below in the Detailed Description. This Summary neither identifies key or essential features, nor limits the scope, of the claimed subject matter.

These challenges can be addressed using corona phase molecular recognition (CoPhMoRe) that enables the molecular recognition of SARS-CoV-2 viral proteins without the need for antibody or enzymatic receptor incorporation.

In one aspect, a sensor can include a nanoparticle structure including a lipid functionalized polyethylene glycol associated with a carbon nanotube.

In another aspect, a method of detecting presence of a coronavirus in a sample can include contacting the sample with a sensor as described herein, and measuring an amount of near infrared fluorescence emitted from the sensor. In certain circumstances, the sample can include saliva. The amount of near infrared fluorescence emitted detector can detect the N protein or the S protein, or both, of the coronavirus at a limit of detection at a concentration between about 40 fM and 400 pM, for example, 48 fM and 350 pM.

In another aspect, a method of developing a sensor for a virus can include selecting a polymer screening array based on one or more structural features of the virus, assembling a plurality of test sensors by associating each member of the polymer screening array with a carbon nanotube, assessing a response of each of the plurality of test sensors to the one or more structural features of the virus, and selecting the sensor based on the response.

In certain circumstances, the lipid functionalized polyethylene glycol can be a phospholipid functionalized polyethylene glycol.

In certain circumstances, the lipid functionalized polyethylene glycol can be a polyethylene glycol-phospholipid heteropolymer.

In certain circumstances, the lipid functionalized polyethylene glycol can have the formula:

(R₁ [symbol] ₙL-Polymer

wherein:

Polymer is a polyethylene glycol;

L is a linking group including an ester, an ether, a phosphate, a thioester, a sulfide, a sulfoxide, a sulfate, a phosphonate, a carbonate, a carbamate, or a carbamide group;

n is 1, 2, or 3; and

each R₁ is, independently, a C6-C24 alkyl chain.

In certain circumstances, the polyethylene glycol can have a terminal group selected from an ether, an ester, a carboxylic acid, or an amine.

In certain circumstances, the polyethylene glycol can have an average molecular weight of between 200 and 10,000. For example, the polyethylene glycol can have an average molecular weight of about 500, 1000, 2000, 3000, 4000, or 5000.

In certain circumstances, each R₁ can be, independently, a C8, C10, C12, C14, C16, C18. C20, C22, or C24 alkyl chain.

In certain circumstances, the lipid functionalized polyethylene glycol is selected from the group consisting of compounds of structures (i) to (xi):

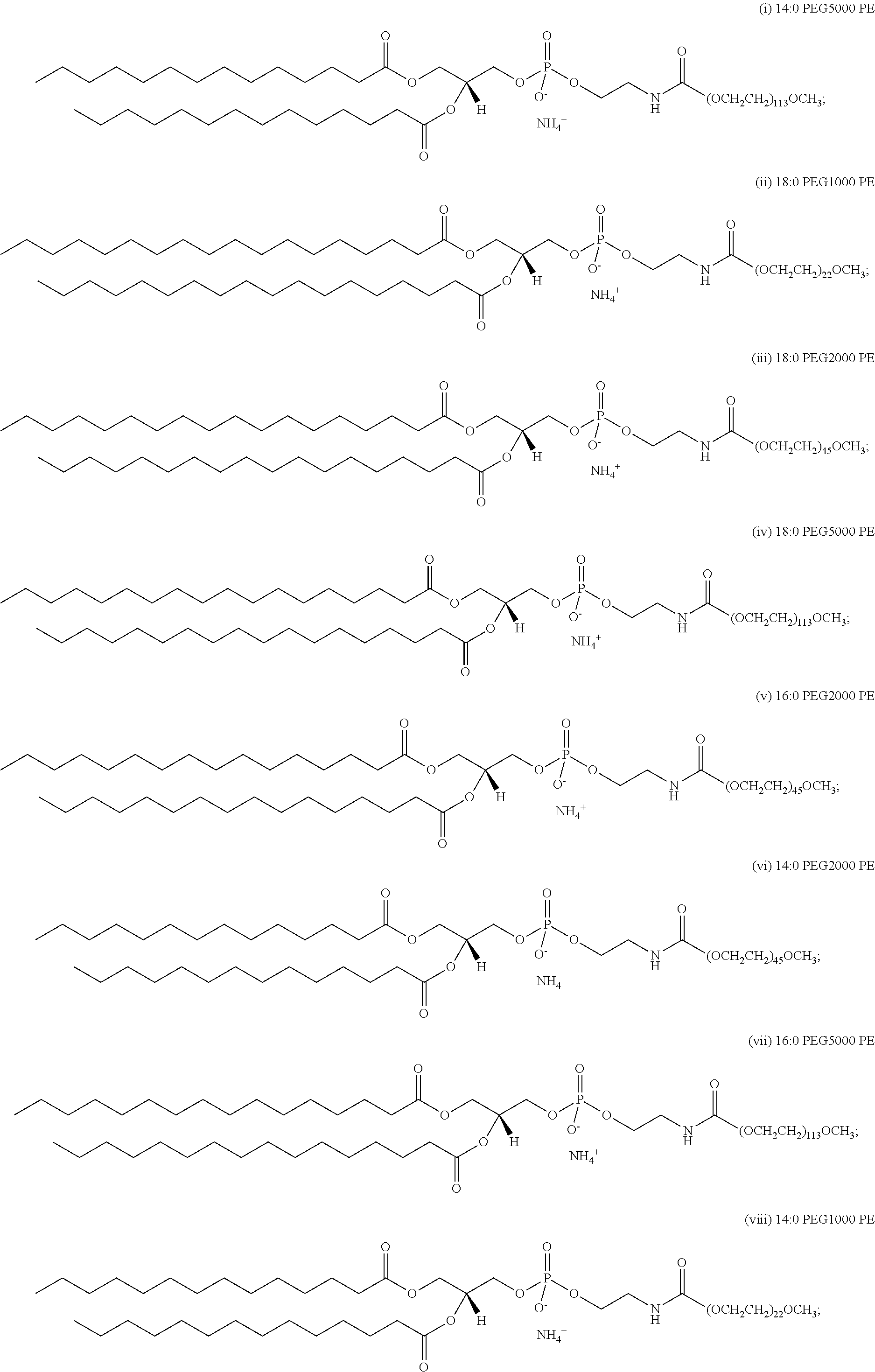

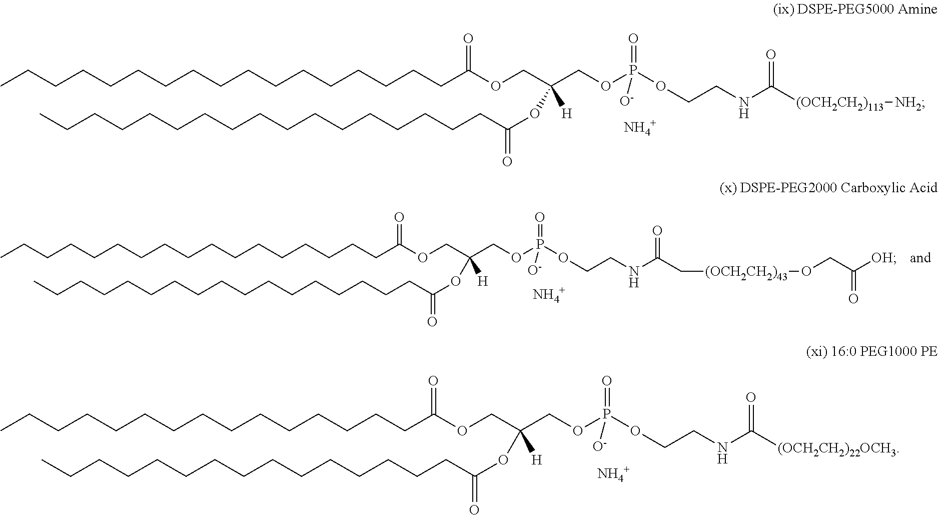

In certain circumstances, the carbon nanotube can be a single-walled carbon nanotube. For example, the single-walled carbon nanotube having a diameter of about 0.8 to 1.2 nm.

In certain circumstances, the carbon nanotube can have a near infrared fluorescence that modulates in the presence of an analyte.

In certain circumstances, the analyte can include a nucleocapsid (N) protein or a spike (S) protein of a coronavirus, or a combination thereof.

In certain circumstances, the coronavirus can include a SARS-CoV-2 type virus.

In certain circumstances, the sensor can be configured to detect the S protein and the lipid functionalized polyethylene glycol comprises the structure of compound (vi)

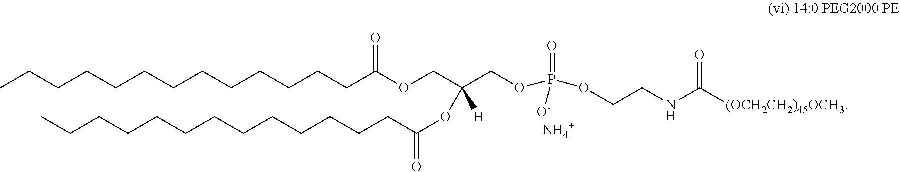

In certain circumstances, the sensor can be configured to detect the N protein and the lipid functionalized polyethylene glycol comprises the structure of compound (ii)

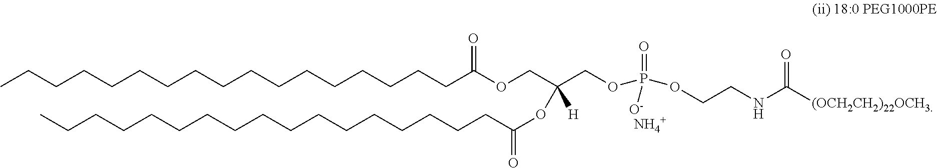

In certain circumstances, the sensor can include an excitation source and an emission detector.

In certain circumstances, the sensor can include a three-dimensional sensing tip with an optical connection for the excitation source and the emission detector.

In certain circumstances, the sensor can include a microscope system including the excitation source and the emission detector.

This recognition nanosensor motif described herein can include specifically selected polyethylene glycol (PEG)-phospholipid heteropolymers adsorbed onto and structured by an underlying single-walled carbon nanotube (SWCNT), whereby a unique three-dimensional (3D) nanoparticle interface recognized nucleocapsid (N) and spike (S) protein of SARS-CoV-2 virus, resulting in rapid and label-free modulation of SWCNT near infrared (nIR) fluorescence. Surprisingly, the presence of the N and S protein of SARS-CoV-2 can elicit a robust and rapid nanosensor fluorescence changes up to 50% and 40% within 5 minutes of analyte injections, and the limits of detection were measured to be 48 fM and 350 pM, respectively. The nanosensor stability was characterized in 100% human saliva condition and demonstrate that N protein sensing ability is completely preserved. Finally, on-site diagnosis system is demonstrated with a fiber optic (optode) benchtop platform that interfaces the advantages of antibody-free molecular recognition virus sensors on a 3D sensing tip.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIGS. 1A-1D 1 illustrates SARS-CoV-2 protein nanosensor library. FIG. 1A is a schematic showing a PEG-phospholipid library for CoPhMoRe based SWCNT nanosensors. FIG. 1B is a graph showing accessible surface area of the PEG-phospholipid wrapped SWCNTs, where q is the vacant binding site on SWCNT, and K_{d} is the dissociation constant of probe binding to SWCNT. Assuming equivalent probe binding strength K_{d}, a higher q/K_{d} value represents more accessible surface area and less corona phase coverage. FIG. 1C is a graph showing UV-vis-nIR absorption spectra of the PEG-phospholipid/SWCNT nanosensor library with distinct E₁₁ and E₂₂ transitions. FIG. 1D is a graph showing nIR fluorescence spectra of the PEG-phospholipid/SWCNT nanosensor library under 785 nm excitation.

FIGS. 2A-2B illustrates SARS-CoV-2 protein nanosensor characterizations. FIG. 2A shows schematics of CoPhMoRe mechanism of PEG-phospholipid/SWCNT nanosensors on N and S viral proteins. FIG. 2B shows a graph of screening results of the integrated normalized response of nanosensors library on N protein (top) and S protein (bottom). Dashed lines indicate best nanosensor for each protein. The SWCNT and protein concentration (N protein, S protein, BSA) were 0.5 mg/L and 10 μg/mL, respectively. N protein buffer: 15 mM Na₂HPO₄, 5 mM NaH₂PO₄, 0.25 M NaCl, pH 7.5, S protein buffer: 2 mM Tris, 200 mM NaCl, pH 8.0. Data are mean (bar)±σ (error bar), with n=3 replicates.

FIGS. 3A-3D illustrates a Limit of Detection (LOD) characterization of two SARS-CoV-2 protein nanosensors for N and S. Fluorescent emission spectra of (FIG. 3A) nanosensor ii and (FIG. 3B) nanosensor vi on wide concentration range (10² fg/mL to 10¹⁰ fg/mL) of N and S protein, respectively. Integrated fluorescence intensity at reaction time=60 min for (FIG. 3C) nanosensor ii and (FIG. 3D) nanosensor vi with cooperative binding model fitting to quantify nanosensor kinetic parameters. Fitting parameters are α=61.91±1.267 and 0.683±0.092, β=3.14±0.852 and −0.005±0.009, K_{D}=0.840±0.121 and 0.213±0.071 nM, and n=1.176±0.101 and 1.120±0.197 for N and S protein, respectively. Data are mean (circle)±σ (error bar), with replicates n=3.

FIGS. 4A-4D illustrate a Lab-on-Fiber on-site monitoring system of SARS-CoV-2 proteins based on nanosensor development in this work. FIG. 4A depicts graphs showing normalized sensor responses of (left) nanosensor ii and (right) nanosensor vi to N and S proteins in buffer, saliva 1%, and saliva 100%. The SWCNT and protein concentration were 0.5 mg/L and 10 μg/mL, respectively. The data represent the mean value of n=3 replicates. FIG. 4B is a graph depicting that nIR fluorescence spectra of the N protein nanosensor shows almost identical responses in buffer and the 100% human saliva condition. FIG. 4C are photo-images of the fully-integrated fiber optic benchtop instrument with benchtop mobile cart. Sensor response to viral protein dropping were real-time measured under 561 nm laser excitation. FIG. 4D is a graph showing real-time fiber optic monitoring of SARS-CoV-2 viral protein using CoPhMoRe nanosensor (10 μg/mL N protein, 5 μL droplet). Top: protein in buffer solution. Bottom: protein in saliva 100%.

FIGS. 5A-5B illustrate potential workflow for CoPhMoRe-based virial detection for a hypothetical future pandemic. FIG. 5A shows an estimated the labor scheduling for such a workflow, the timeline for the SARS-CoV-2 protein sensor development of the work described herein. Approximately 10 days of laboratory effort from two researchers with approximately 4 hours per day completed the development. This includes experiments with associated waiting and analysis time, excluding reagent delivery time. FIG. 5B is a schematic of an accelerated point-of-care sensor development with continual feedback to address emerging viral targets in the future.

FIG. 6 is a graph depicting a molecular probe adsorption method for SWCNT surface coverage measurement, with slope inversely proportional to the accessible surface area ranked in descending order.

FIG. 7A is a graph depicting fluorescent emission spectra of nanosensor ii and nanosensor vi on control buffer, BSA, and viral protein.

FIG. 7B is a graph depicting real-time fluorescence variations of nanosensors ii and nanosensor vi on N protein and S protein, respectively.

FIGS. 8A-8B a graphs showing normalized change in fluorescence of the 1125 nm emission peak corresponding to (7, 6) chirality SWCNT of (FIG. 8A) nanosensor ii and (FIG. 8B) nanosensor vi as a function of time over 60 min.

### DETAILED DESCRIPTION

Sensors, also referred to herein as nanosensors, can also support new molecular recognition technologies, such as CoPhMoRe (Corona Phase Molecular Recognition), that generate synthetic, non-biological recognition sites. Antibody or enzymatic receptor-free nanosensors developed using this technology can rapidly identify one or more viral proteins in a sample. The approach to developing the nanosensors uses several strategies to design selective molecular sensors using near-infrared (NIR) fluorescent single-walled carbon nanotubes (SWNTs). Upon molecular recognition and analyte binding, the SWNT fluorescence signal is modulated and optically measured via an infrared (IR) detector, for example a near IR (nIR) detector. The rapid optical readout and long-term fluorescence stability of the SWNT sensors allow for scaling of the label-free technology for a high degree of multiplexation. The ability to collect dynamic real-time binding data provides a significant advantage over conventional fluorescence-based detection platforms.

In one aspect, a sensor can include a nanoparticle structure including a lipid functionalized polyethylene glycol associated with a carbon nanotube.

The sensor described herein can be used in a fiber optic-based system. This can allow for a small form factor that is portable and can easily be integrated into different areas of the drug processing steps. This technique increases the portability and decreases the size of the ultimate form factor.

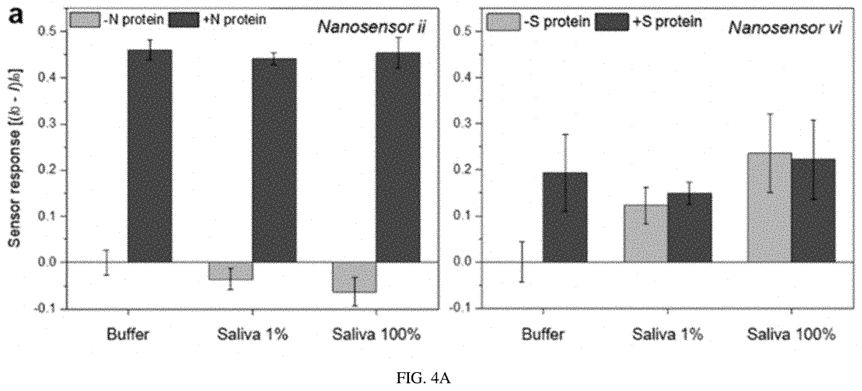

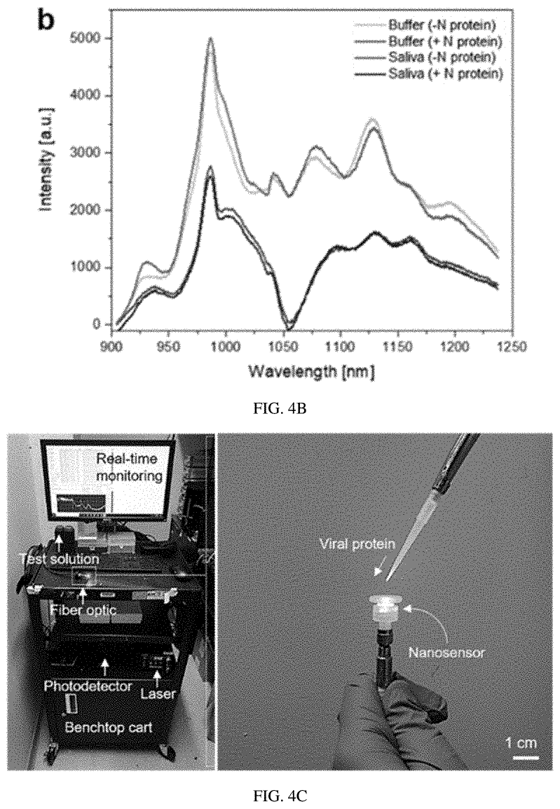

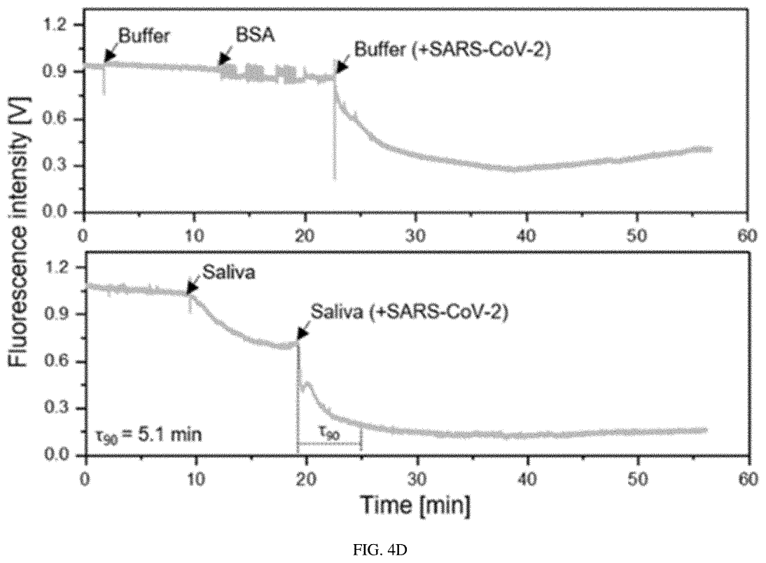

A detection system can be a include a housing including a chamber, a light port and a sample contact port, and the sensor including the nanoparticle structure, the nanoparticle structure configured to interact with a sample, the sensor having a surface in contact with the light port and a sample contact surface adjacent to the sample contact port. See, for example, FIG. 4C, which depicts an exemplary the housing, light port, and sample contact port. The nanoparticle structure can be supported in or on a hydrogel. Alternatively, the nanoparticle structure can be supported in or on a solid support. In certain circumstances, the nanoparticle structure can be in a liquid.

A sensor can be a portion of a device including a housing including a chamber, a light port and a sample contact port; and a fiber optic including an excitation fiber configured to provide an excitation wavelength and a detection fiber configured to detect an emission wavelength. The chamber is configured to contain a composition including the sensor described herein. The fibers can be housed in a multi-fiber housing so that the detection fiber is proximate to the excitation fiber. The multi-fiber housing can include 1, 2, 3, 4, 5, 6, 7, or 8 excitation fibers. The multi-fiber housing can include 1, 2, 3, 4, 5, 6, 7, or 8 detection fibers. The detection fibers can encircle the excitation fiber (or excitation fibers). For example, 2, 3, 4, 5 or 6 detection fibers can surround a single excitation fiber.

In certain circumstances, the nanoparticle structure can be supported in or on a matrix, for example, in or on a hydrogel. The hydrogel can be a polymeric hydrogel. For example, the hydrogel can be agarose, a polyalcohol, or other matrix polymer. The hydrogel can be any matrix that allows for diffusion of the analyte. Alternatively, the nanoparticle structure can be suspended in a liquid or on a porous substrate. The matrix can include a polyvinyl chloride, a polyethylene, a polyester, a polypropylene, a polycarbonate, a polyacrylamide, or a polyvinyl alcohol.

In certain circumstances, the light port can be configured to attach to a fiber optic.

In certain circumstances, the fiber optic can include an excitation fiber configured to provide an excitation wavelength to the nanostructure. In certain circumstances, the fiber optic can include a detection fiber configured to detect an emission wavelength from the nanostructure. For example, the excitation fiber can be the light delivery fiber and the detection fiber can be the light detection fiber shown in an exemplary sensor device.

A sensor can include a nanoparticle structure. A nanoparticle structure can include an emissive nanostructure, which can have at least one cross-sectional dimension between opposed boundaries of less than about 1 micron. In some embodiments, a nanoparticle structure can have at least one cross-sectional dimension between opposed boundaries of less than about 500 nm, less than about 250 nm, less than about 100 nm, less than about 75 nm, less than about 50 nm, less than about 25 nm, less than about 10 nm, or in some cases, less than about 1 nm.

Examples of an emissive nanostructure can include a nanotube (including a carbon nanotube), a nanowire (including a carbon nanowire), a nanorod, a nanofiber, graphene or a quantum dot, among others. An emissive nanostructure can include a fullerene, for example, a carbon nanotube, a buckyball, a buckytube, or a fullerene ring. A nanostructure can also include a nanocrystal. An emissive nanostructure can include a metal, a nonmetal, or semiconductor.

An emissive nanostructure can be a photoluminescent nanostructure, which can exhibit photoluminescence. See, for example, U.S. Pat. No. 10,215,752, which is incorporated by reference in its entirety. In some instances, photoluminescent nanostructures can exhibit fluorescence. For example, a photoluminescent nanostructure can emit fluorescence with a wavelength in the near infrared spectrum. In some instances, photoluminescent nanostructures can exhibit phosphorescence. A photoluminescent nanostructure can be a nanotube. A nanotube can be a carbon nanotube. A carbon nanotube can be a single walled carbon nanotube. In some embodiments, a photoluminescent nanostructure can be a semi-conductive single-walled carbon nanotube. Additional examples of photoluminescent nanostructures can include, but are not limited to, double-walled carbon nanotubes, multi-walled carbon nanotubes, semi-conductor quantum dots, semi-conductor nanowires, or graphene, among others.

An emissive nanostructure can have a property that can be altered by changes in the environment of the emissive nanostructure. The property can be detectable or observable. The property can also be measurable so that changes in the property can be described or quantified. The property can be photoluminescence, conductivity, polarity, or resonance. Photoluminescence can be fluorescence or phosphorescence. The photoluminescence can be fluorescence with a wavelength within the near infrared spectrum. A property can be an emission wavelength, an emission intensity, a conductance, an electromagnetic absorbance or an emittance.

If the emissive nanostructure is a carbon nanotube, the carbon nanotube can be classified by its chiral vector (n,m), which can indicate the orientation of the carbon hexagons. The orientation of carbon hexagons can affect interactions of the nanotube with other molecules, which in turn, can affect a property of the emissive nanostructure. In certain embodiments, the carbon nanotube is a single-walled carbon nanotube having a (6, 5), (7, 6), or (9, 4) nanotube chirality. A plurality of carbon nanotubes can include a combination of chiralities.

In certain circumstances, the carbon nanotube can be a single walled carbon nanotube having a diameter of greater than about 0.5 nm, greater than about 0.6 nm, greater than about 0.7 nm, or greater than about 0.8 nm. In certain circumstances, the carbon nanotube can be a single walled carbon nanotube having a diameter of less than about 2.0 nm, less than about 1.8 nm, less than about 1.6 nm, or greater than about 1.4 nm. For example, the single walled carbon nanotube can have a diameter of between about 0.8 nm and about 1.2 nm.

A nanoparticle structure can exhibit solvatochromism. Analytes that change the local dielectric constant can change the photoluminescence of the emissive nanostructure. An interaction of an electron-donating or -withdrawing molecule with a nanoparticle structure can alter a property, for example photoluminescence, of the emissive nanostructure. An interaction with a nanoparticle structure can be direct or indirect. Additionally, more than one electron-donating or -withdrawing molecule can interact with an emissive nanostructure and each molecule can alter a property of the emissive nanostructure. A second molecule can also interact with an electron-donating or -withdrawing molecule and change the relationship of the electron-donating or -withdrawing molecule to the emissive nanostructure. This can also alter an emissive nanostructure property. For example, a first molecule can interact with the emissive nanostructure and alter a property (e.g. the photoluminescence) of the emissive nanostructure, and then a second molecule can interact with either the emissive nanostructure or the first molecule and further alter a property (e.g. the photoluminescence) of the emissive nanostructure.

The change in the property can be caused by a change in the distance between an ion in the first binding partner and the nanostructure. As the distance between the nanostructure and the ion changes, a nanostructure property can also change. For example, as the distance between the nanostructure and the ion changes, nanostructure photoluminescence can also change. When the capture protein binds to the linker, the distance between the ion and nanostructure can change, which can alter the nanostructure photoluminescence. Generally, as the distance between the ion and the nanostructure decreases, the amount of photoluminescence quenching can increase.

A lipid functionalized polyethylene glycol can be associated with the emissive nanostructure. The association can be a bond, for example, a covalent, ionic, van der Waals, dipolar or hydrogen bond. The association can be a physical association. For example, at least a portion of the emissive nanostructure can be embedded in the polymer or a portion of the polymer can encompass the emissive nanostructure.

The association of a lipid functionalized polyethylene glycol with a nanostructure can change a property of the emissive nanostructure. The property can be conductivity, polarity, or resonance. The property can be photoluminescence, including fluorescence or phosphorescence. More specifically, the property can be fluorescence with a wavelength in the near infrared spectrum. The property can be an emission wavelength, an emission intensity, a conductance, an electromagnetic absorbance or an emittance.

A lipid functionalized polyethylene glycol can include functionalized polyethylene glycol. The functionalized polyethylene glycol can be a polyethylene glycol having a molecular weight (i.e., a weight average molecular weight or a number average molecular weight) of between 100 and 100,000 daltons, for example, between 200 and 10,000 daltons. In certain circumstances, the polyethylene glycol can have an average molecular weight of about 500, 750, 1000, 1500, 2000, 2500, 3000, 3500, 4000, 4500, 5000, 5500, 6000, 6500, 7000, 7500, 8000, 8500, 9000, 9500, or 10000 daltons. The polyethylene glycol can have a terminal group selected from an ether, an ester, a carboxylic acid, or an amine. The ether can be a C1-C6 ether, for example, methyl ether, ethyl ether, propyl ether, butyl ether, pentanyl ether, or hexanyl ether. The ester can be a methyl ester or ethyl ester.

The lipid functionalized polyethylene glycol can have one, two, three or more lipid chains covalently bonded to a terminal group of the polyethylene glycol. For example, the lipid chain can be opposite the terminal group selected from an ether, an ester, a carboxylic acid, or an amine.

Each lipid chain can be independently selected from the group consisting of a C6-C24 alkyl chain. Preferably, the alkyl chain can be a C8, C10, C12, C14, C16, C18, C20, C22, or C24 alkyl chain. The alkyl chain can be optionally interrupted by one or more carbon-carbon double bonds or carbon-carbon triple bonds. The alkyl chain can be branched or unbranched.

In certain embodiments, the lipid functionalized polyethylene glycol can have the formula:

(R₁ [symbol] L-Polymer

wherein:

Polymer is a polyethylene glycol;

L is a linking group including an ester, an ether, a phosphate, a thioester, a sulfide, a sulfoxide, a sulfate, a phosphonate, a carbonate, a carbamate, or a carbamide group;

n is 1, 2, or 3; and

each R₁ is, independently, a C6-C24 alkyl chain.

Preferably, n is 1 or 2.

Preferably, the polyethylene glycol can have an average molecular weight of about 500, 1000, 2000, 3000, 4000, or 5000.

Preferably, each R₁ can be, independently, a C8, C10, C12, C14, C16, C18, C20, C22, or C24 alkyl chain.

In certain embodiments, the linking group can be a linking moiety including a saturated or unsaturated C₄₋₁₀ hydrocarbon chain optionally containing at least two conjugated double bonds, at least one triple bond, or at least one double bond and one triple bond; said hydrocarbon chain being optionally substituted with C₁₋₄alkyl, C₂₋₄ alkenyl, C₂₋₄ alkynyl, C₁₋₄ alkoxy, hydroxyl, halo, carboxyl, amino, nitro, cyano, C₃₋₆ cycloalkyl, 3-6 membered heterocycloalkyl, unsubstituted monocyclic aryl, 5-6 membered heteroaryl, C₁₋₄ alkylcarbonyloxy, C₁₋₄ alkyloxycarbonyl, C₁₋₄ alkylcarbonyl, or formyl and said hydrocarbon chain being optionally interrupted by one or more of O, S, S(O), S(O)₂, OS(O)O, OS(O)₂O, P(R^{a}), P(R^{a})(O), OP(O)₂⁻O, N(R^{a}), C(O), N(R^{a})C(O)O, OC(O)N(R^{a}), N(R^{a})C(O)N(R^{b}), C(O)O, OC(O)O; each of R^{a} and R^{b}, independently, being hydrogen, alkyl, alkenyl, alkynyl, alkoxy, hydroxylalkyl, hydroxyl, or haloalkyl, or L can be a bond, and C can be a metal ion complexing moiety.

Note that an amino group can be unsubstituted (i.e., —NH₂), mono-substituted (i.e., —NHR), or di-substituted (i.e., —NR₂). It can be substituted with groups (R) such as alkyl, cycloalkyl, heterocycloalkyl, aryl, heteroaryl, aralkyl, or heteroaralkyl. Halo refers to fluoro, chloro, bromo, or iodo.

In some circumstances, L can be a glycero-3-phosphoethanolamine group.

In yet another preferred embodiment, the lipid functionalized polyethylene glycol can be a polyethylene glycol-phospholipid heteropolymer.

For example, the lipid functionalized polyethylene glycol compound can have the formula:

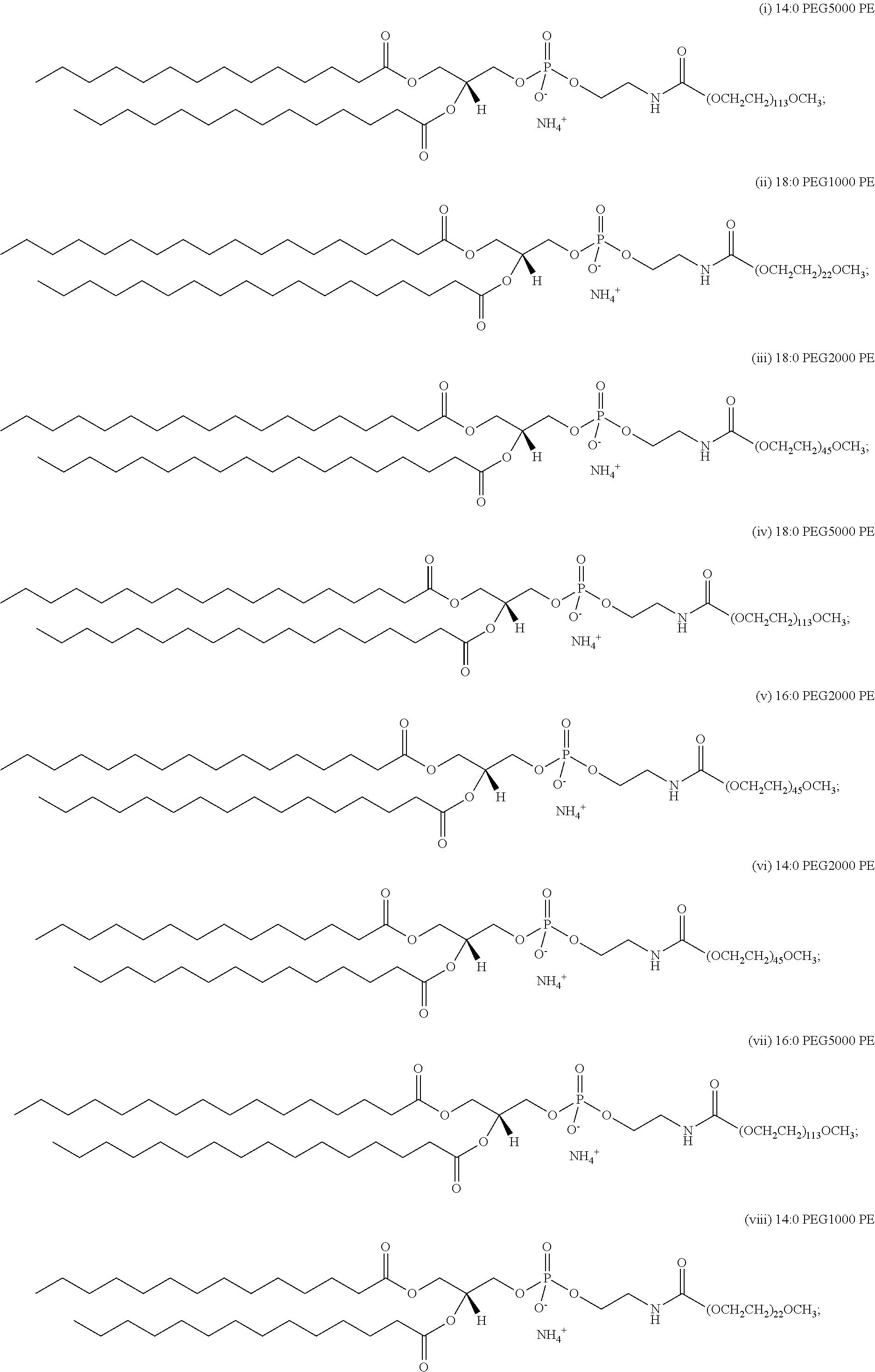

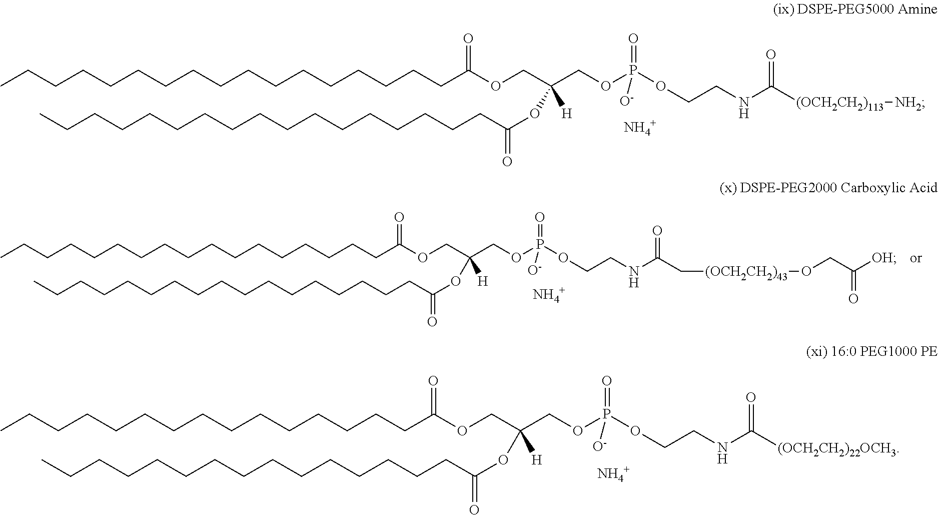

Compounds can be prepared according to published procedures such as those described, for example, in Parameswara et al., Synthesis, 815-818 (1980) and Denny et al., J. Org. Chem., 27, 3404 (1962), which is incorporated by reference in its entirety.

In another aspect, an array can include a plurality of analysis regions on a substrate. A substrate can be glass or plastic.

An analysis region can be a divot, a tube, a tray, a well or a similar compartment for suitable for containing a liquid sample. In some cases, an analysis region can include a droplet or spot on the surface of a substrate. In those cases, an analysis region can be formed by spotting a composition on a substrate. A plurality of analysis regions can be in a pattern on a substrate. A pattern can include concentric circles, a spiral, a row, a column or a grid.

In certain circumstances, the sensor can detect a component of a sample. The sample can include a gas, a liquid or a solid. In some embodiments, the sample can be a biological fluid, for example, saliva or mucus.

In certain circumstances, the sensor including the carbon nanotube has a near infrared fluorescence that modulates in the presence of an analyte in the sample. The analyte can include a nucleocapsid (N) protein or a spike (S) protein of a coronavirus, or a combination thereof. For example, the sensor can include a first nanoparticle structure including a lipid functionalized polyethylene glycol associated with a carbon nanotube that has an emission responsive to a nucleocapsid (N) protein. In another example, the sensor can include a second nanoparticle structure including a lipid functionalized polyethylene glycol associated with a carbon nanotube that has an emission responsive to a spike (S) protein. In another example, the sensor can include a first nanoparticle structure including a lipid functionalized polyethylene glycol associated with a carbon nanotube that has an emission responsive to a nucleocapsid (N) protein and a second nanoparticle structure including a lipid functionalized polyethylene glycol associated with a carbon nanotube that has an emission responsive to a spike (S) protein.

In certain circumstances, the coronavirus can include a SARS-CoV-2 type virus.

In certain circumstances, the sensor can be configured to detect the S protein and the lipid functionalized polyethylene glycol comprises the structure of compound (vi)

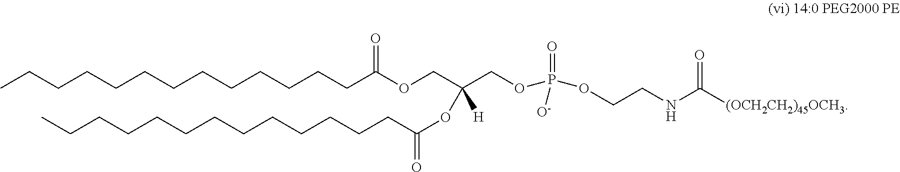

In certain circumstances, the sensor can be configured to detect the N protein and lipid functionalized polyethylene glycol comprises the structure of compound (ii)

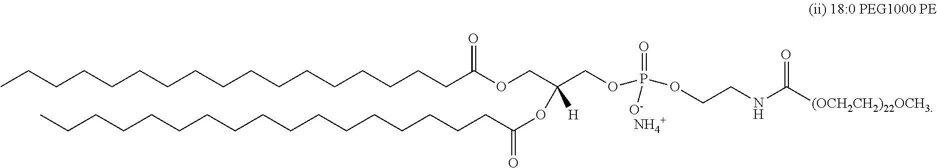

In certain circumstances, the sensor can include an excitation source and an emission detector. The emission detector can be a near infrared emission detector.

In certain circumstances, the sensor can include a three-dimensional sensing tip with an optical connection for the excitation source and the emission detector as described herein, optionally with a fiber optic. In certain circumstances, the sensor can include a microscope system including the excitation source and the emission detector. For example, the sensor can include hardware capable of visible excitation followed by near-infrared emissions measurement can be used to interrogate the sensors. The hardware can include a microscope having objectives, lenses, dichroics, short pass optical filters, long pass optical filters and mechanized stages. Visible excitation can be any sources capable of visible excitation, including LEDs, Lasers and broad spectrum sources. Detectors can be hardware capable of detecting 850-1350 nm, including silicon and/or Indium gallium arsenide detector elements cooled passively, via thermoelectrics or liquid nitrogen.

In another aspect, a method of detecting presence of a coronavirus in a sample can include contacting the sample with a sensor as described herein; and measuring an amount of near infrared fluorescence emitted from the sensor. For example, the sample can include saliva.

In certain circumstances, the amount of near infrared fluorescence emitted detector can detect the N protein or the S protein, or both, of the coronavirus at a limit of detection at a concentration between about 40 fM and 500 pM, for example, between about 48 fM and 350 pM.

In another aspect, a method of developing a sensor for a virus can include selecting a polymer screening array based on one or more structural features of the virus, assembling a plurality of test sensors by associating each member of the polymer screening array with a carbon nanotube, assessing a response of each of the plurality of test sensors to the one or more structural features of the virus, and selecting the sensor based on the response.

In certain circumstances, the polymer screening array can include one or more polymers as described below.

In some embodiments, the polymer can be a polysaccharide. The polysaccharide can include peptidoglycans, lipopolysaccharides, amylase, chitin, chitosan, glycogen, cellulose, dextran, functionalized dextran, phenoxy functionalized dextran or boronic acid functionalized phenoxy dextran.

In some embodiments, the polymer can be a polynucleotide. The polynucleotide can be DNA or RNA. The polynucleotide can be single stranded or double stranded. The polynucleotide can be single stranded in one section and double stranded in another section. RNA can include mRNA, siRNA or shRNA.

The polynucleotide can form a structure. Exemplary nucleic acid structures can include an A-form double helix, a B-form double helix, a Z-form double helix, a hairpin, a loop or a stem loop.

The polynucleotide can contain ribonucleotides or deoxyribonucleotides. The polynucleotide can have less than 100,000, less than 50,000, less than 25,000, less than 10,000, less than 5,000, less than 1,000, less than 500, less than 250, less than 100, less than 75, less than 50, less than 30, less than 25, less than 20, 15, 12, 10, 8, 6 or 4 nucleotides.

The polynucleotide can have a random sequence. The polynucleotide can have an ordered sequence. The ordered sequence can be a predetermined sequence. For example, an ordered sequence can be the sequence of a gene. The ordered sequence can be a repeating sequence. The repeat sequence can include less than 500, less than 400, less than 300, less than 200, less than 100, less than 50, less than 30, less than 25, less than 20, 15, 14, 13, 12, 11, 10, 9, 8, 7, 6, 5, 4, 3 or 2 nucleotides. The polynucleotide can be poly(AT), poly(GT), poly(CT), poly(AG), poly(CG), or poly(AC). The polynucleotide can have a content. The content can be a percentage of a unique nucleotide present in the sequence. The percentage can be 100% of a unique nucleotide, including poly(A), poly(C), poly(G), poly(T) or poly(U).

In some embodiments, the polymer can be a polylipid. The polylipid can include a phospholipid, a palmitoyl phospholipid or 1,2-dipalmitoyl-sn-glycero-3-phosphoethanolamine-N-(lauroyl) (PL-DOD).

In other embodiments, the polymer can be polyvinylpyrrolidone, a poly(ethylene oxide), a poly(ethylene oxide)-poly (propylene oxide)-poly (ethylene oxide) block co-polymer, poly (N-isopropyl acrylamide), polyethyleneimine, polyacrylamide, polyvinyl alcohol or collagen.

In some embodiments, the polymer can be a polypeptide. In some embodiments, the number of amino acids comprising the polypeptide can fall within a specific range. For example, the polypeptide can include between 5 and about 50 amino acid residues, or between 5 and about 30 amino acid residues. In other embodiments, the polypeptide can fall within a specific molar mass range. For example, the polypeptide can have a molecular weight of between 400 g/mol and about 10,000 g/mol, or between 400 g/mol and about 6,000 g/mol. The polypeptide can be a protein, having greater than about 50 amino acid residues. The polypeptide can be a fragment of a protein. In some embodiments, the polypeptide can be expressed in a disease state. In other embodiments, the polypeptide can be modified. In some circumstances, the polypeptide can be modified by attaching functional groups. In other circumstances, the polypeptide can be ubiquinated, biotinylated, glycosylated, PEGylated or SUMOylated. The polypeptide can be a biomarker, an enzyme, a receptor, a ligand, a peptide hormone, a neuropeptide, a vasoactive intestinal peptide, a chaperone or an antibody.

The polypeptide can, in some instances, include a peptide sequence observed in the venom of an animal or a derivative thereof. In some cases, the polymer can include a polypeptide sequence (or derivative thereof) observed in the venom of a member of the Insecta class, the Hymenoptera order, or the Vespidae or Apidae families. In some embodiments, the polypeptide can be a member the Mastoparan or Bombolitin (including Bombolitin II, Bombolitin III) families of polypeptides, or derivatives of those polypeptides. The polypeptide can include a mastoparan, mastoparan 7 or mastoparan X.

In some embodiments, the polymer can be a lipid functionalized polyethylene glycol as described above.

The method can include assembling a plurality of test sensors by associating each member of the polymer screening array with a carbon nanotube, for example, by the methods described herein.

The effectiveness of the plurality of test sensors can then be evaluated. For example, by assessing a response of each of the plurality of test sensors to the one or more structural features of the virus. This can lead to selecting the sensor based on the response.

An example of this method is described in the following sections by way of example.

The recognition motif of nanosensors described herein includes specifically selected polyethylene glycol (PEG)-phospholipid heteropolymers adsorbed onto and structured by an underlying single-walled carbon nanotube (SWCNT), whereby a unique three-dimensional (3D) nanoparticle interface recognized nucleocapsid (N) and spike (S) protein of SARS-CoV-2 virus, resulting in rapid and label-free modulation of SWCNT near infrared (nIR) fluorescence. The presence of the N and S protein of SARS-CoV-2 elicited a robust and rapid nanosensor fluorescence changes up to 50% and 40% within 5 min analyte injections, and LOD were measured to be 48 fM and 350 pM, respectively. The nanosensor stability in 100% human saliva condition was characterized and demonstrated that N protein sensing ability is completely preserved. Finally, on-site diagnosis system was demonstrated with a fiber optic (optode) benchtop platform that interfaced the advantages of antibody-free molecular recognition virus sensors on a 3D sensing tip.

An example implementation of techniques for corona phase molecular recognition is provided below.

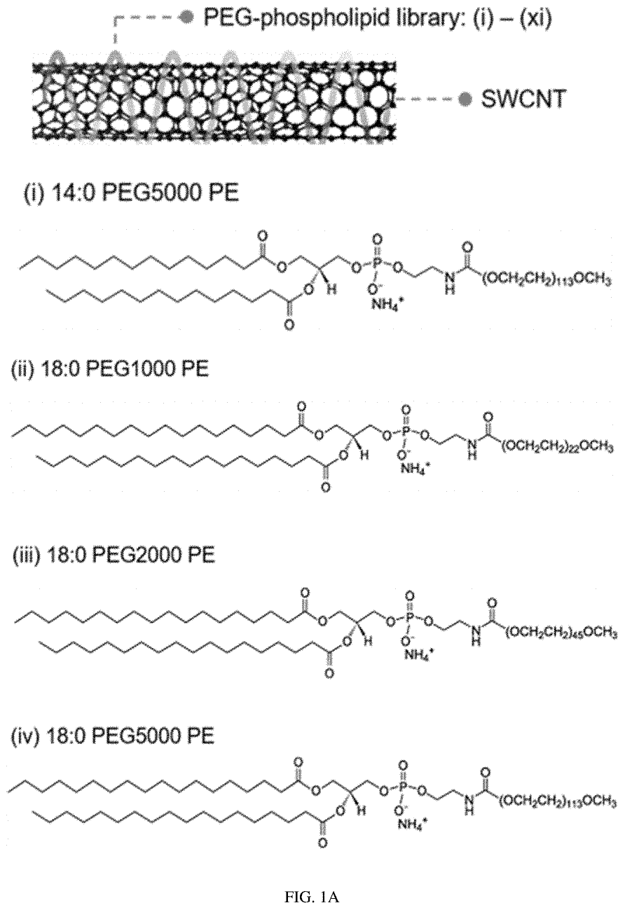

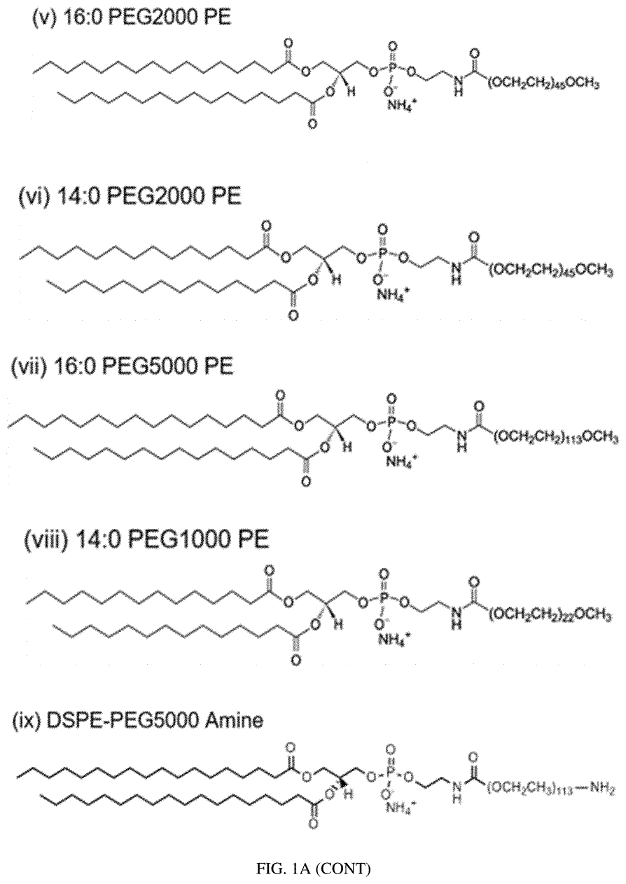

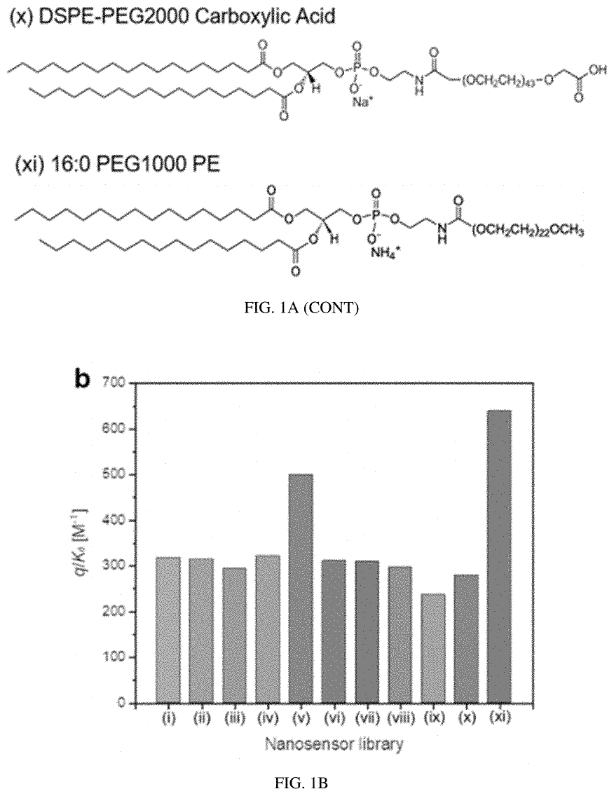

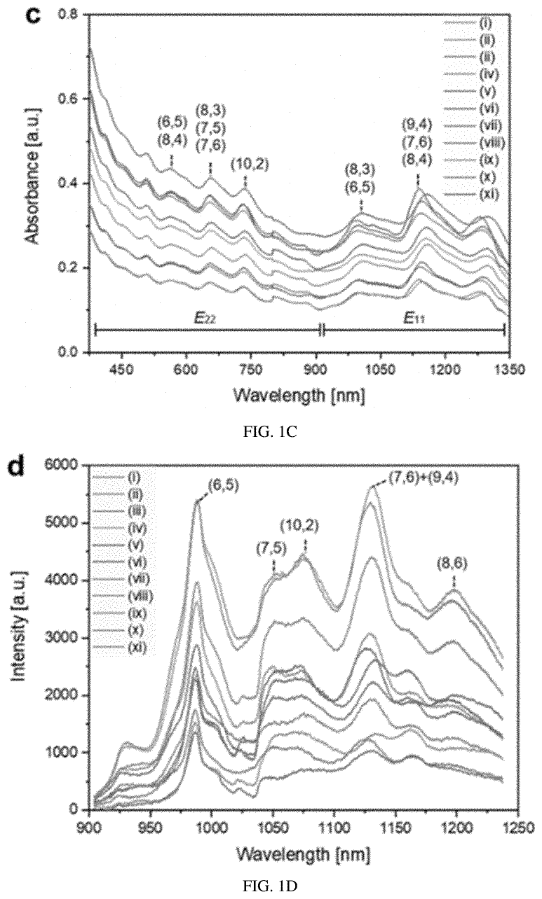

SWCNT synthesized by the high-pressure carbon monoxide (HiPCO) process were suspended with a specifically designed library of 11 PEG-phospholipid polymers capable of forming corona phases at the SWCNT surface (FIG. 1A). PEG-phospholipid polymers are chosen for corona formation since previous studies have shown that PEG-phospholipid wrapped SWCNT were capable of detecting proteins in complex biofluid condition (ref. 31). In addition, chain length of PEG can be easily tuned to form wide range of the binding pocket, and they are commercially available thus, the preparation of polymer library is not time consuming. The accessible surface area of the PEG-phospholipid wrapped SWCNT was measured by titration using a quenchable fluorescent riboflavin probe following a recently developed molecular probe adsorption (MPA) technique (ref. 32), described below. Results show that DSPE-PEG5000 Amine formed the largest surface covering corona on SWCNT, and 16:0 PEG1000 PE formed smallest surface coverage corona (FIG. 1B). The resulting colloidal solutions of nanosensors are characterized by ultraviolet-visible-nIR (UV-vis-nIR) absorption spectroscopy. FIG. 1C shows the UV-vis-nIR absorption spectrum for the PEG-phospholipid corona phase, where the distinct and sharp peaks of E₁₁ and E₂₂ transitions indicate the successful isolation and suspension of individual SWCNT. nIR fluorescent emission spectra (FIG. 1D) under 785 nm laser excitation demonstrate that nanosensors are mainly composed of (6, 5), (7, 6), and (9, 4) nanotube chirality.

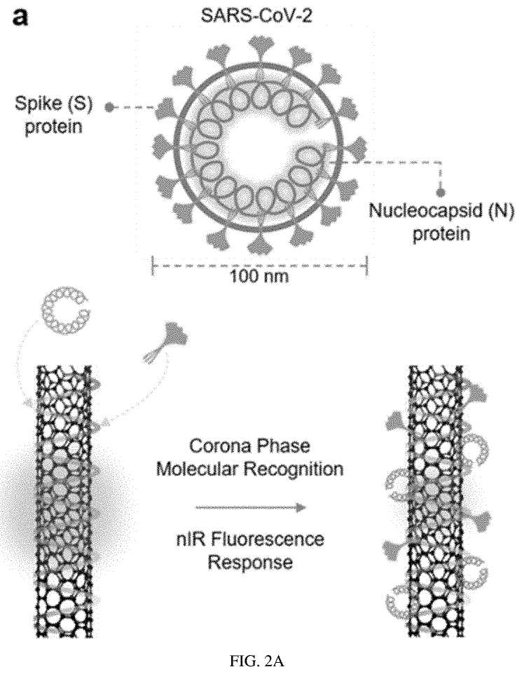

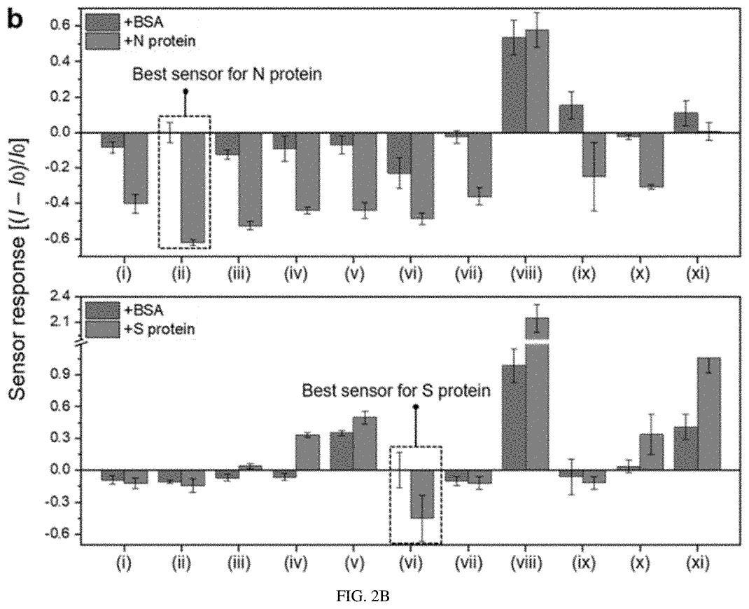

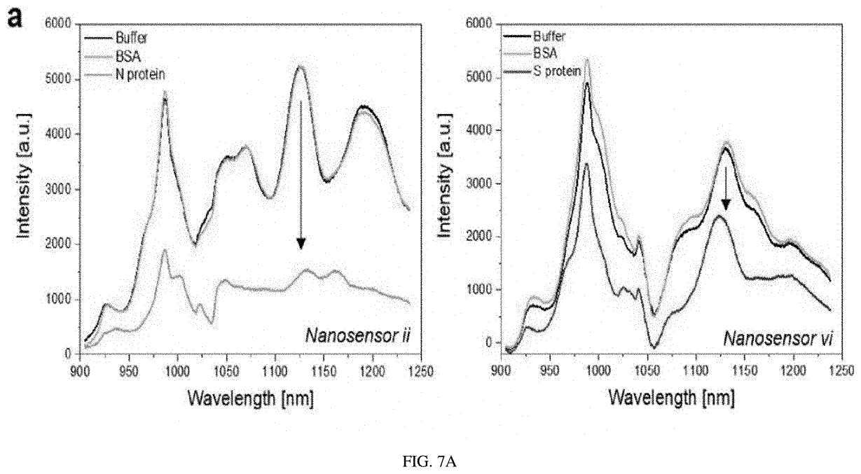

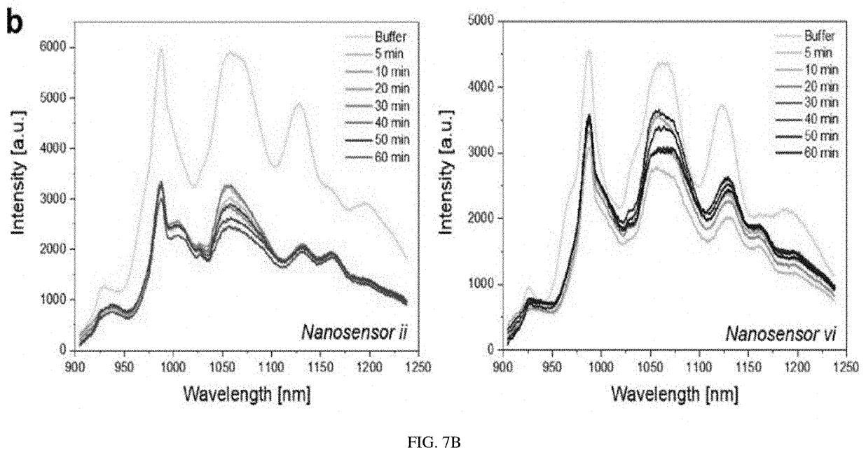

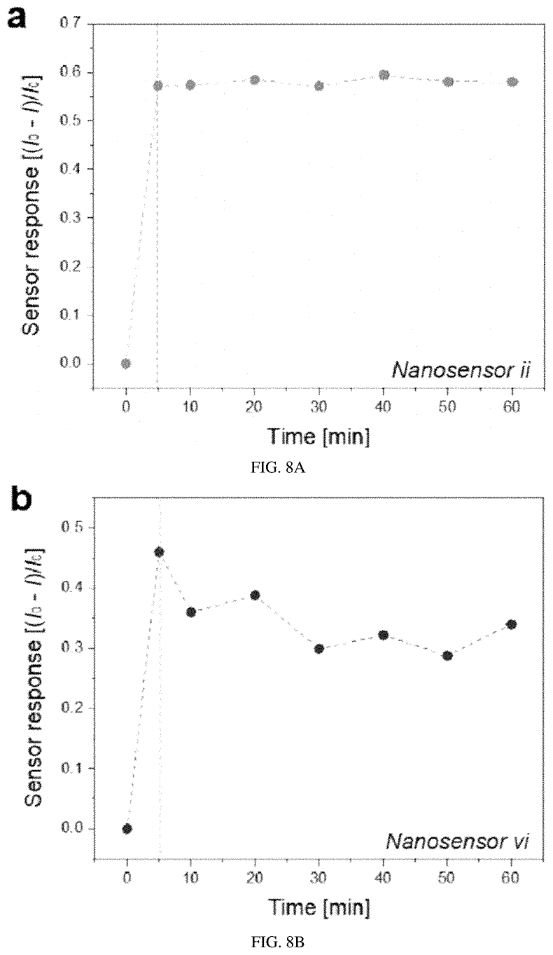

The nIR fluorescence response of PEG-phospholipid/SWCNT nanosensors was measured during SARS-CoV-2 N and S protein addition, schematically represented in FIG. 2A. The N and S proteins can be recognized by specific 3D corona phase configurations of the PEG-phospholipid polymers imposed by the nanoparticle interfaces enabling rapid modulation of underlying nIR fluorescence intensity of the nanotube (refs. 31, 33). In this way, the corona phase (PEG-phospholipid) acts as the receptor coupled directly to the fluorescent nanotube, which acts as the nonselective transducer. Note that specific recognition between the PEG and phospholipid independently of the nanotube surface is not expected. The combined polymer/nanotube construct is labeled CoPhMoRe. The unit composition of the polymers were varied to produce structurally diverse corona phases to sample a range of free-volumes and chemical interactions with the analyte influencing the dynamic binding/unbinding of viral proteins (ref. 34). In total, 44 unique corona phases were explored based on 11 PEG-phospholipid polymer backbones. The responses of the resulting fluorescent emission of this library were recorded following an hour incubation with each of viral protein target at 10 μg/mL in distinct buffer conditions. An identical concentration of strongly adsorbing bovine serum albumin (BSA) was used as an interferent to address both issues of non-specific binding and the stability of the sensor. Resulting bar graph chart of molecular binding shows distinct fluorescence responses ((I−I₀)/I₀) with varying polymer compositions (FIG. 2B). Here, I₀ and I represent the integrated nIR intensity of nanosensors at t=0 and after viral proteins injection, respectively. Each nanosensor shows a unique turn-on or turn-off response to N proteins, S proteins, and BSA with response intensity changes from −70% to 212%. Most of the sensors demonstrate quenching or turn-off responses to N protein and intensity gains or turn-on responses to S protein. The 18:0 PEG1000 PE/SWCNT (henceforth labeled nanosensor ii) and 14:0 PEG2000 PE/SWCNT (nanosensor vi) show the most obvious and clearly distinguishable responses to N protein and S protein respectively with strong 50 to 70% decreases in fluorescence intensity. Importantly, BSA appears to induce almost no response for these corona phase complexes. Thus, for the purposes of this work, nanosensor ii and nanosensor vi can be considered specific sensors for N protein and S protein, respectively. Fluorescent emission spectra demonstrate that nanosensor ii and nanosensor vi show significant nIR intensity decreases only to their specific protein targets (FIG. 7A). Time-series emission spectra show that intensities of both nanosensors do not change after 60 min of buffer incubation; however, they drastically change within 5 min of the addition of viral proteins (FIG. 7B). More specifically, the normalized change in the fluorescence of the 1125 nm emission peak corresponding to (7, 6) chirality SWCNT instantaneously decreased by 57 and 45% within 5 min (FIGS. 8A-8B) for nanosensor ii and nanosensor vi, respectively. The fluorescent response of nanosensor ii to N protein maintained for 60 min and response of nanosensor vi decrease after first peak response of S protein.

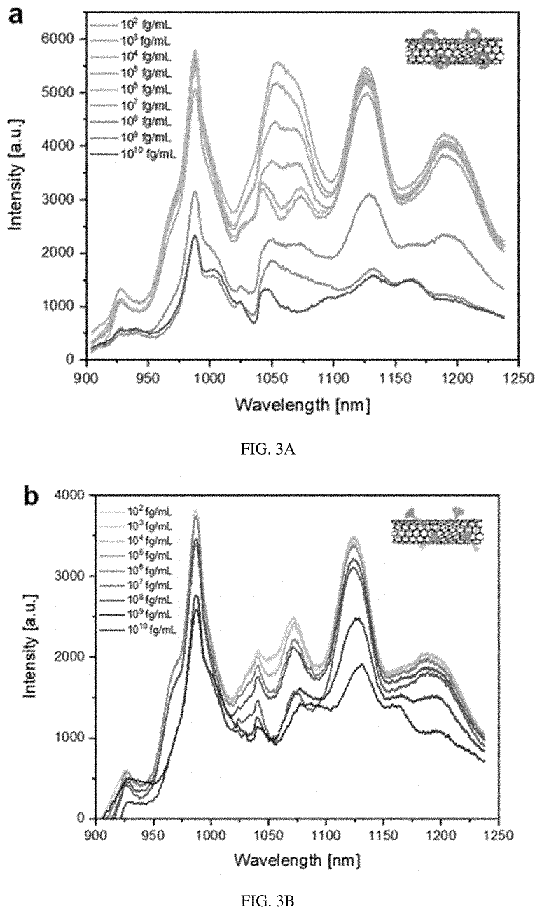

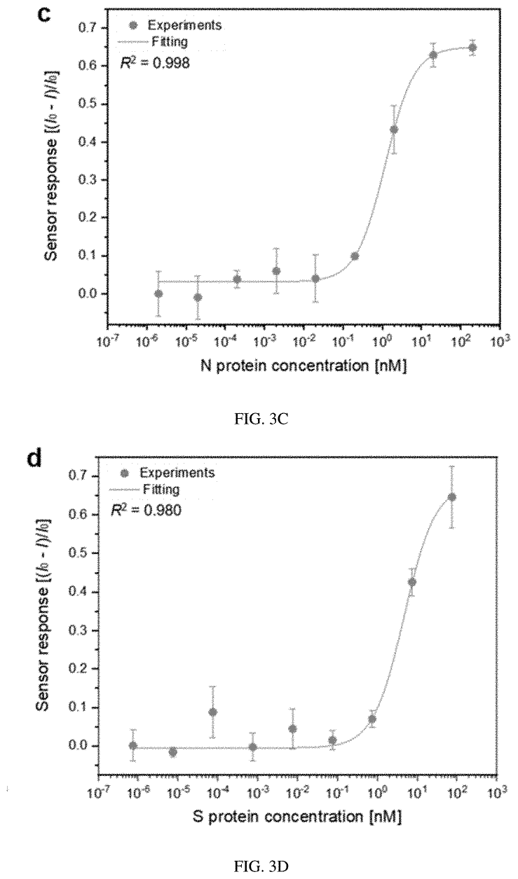

Real-time nIR sensor responses were measured using a wide range of concentrations from the fM to nM level of N and S proteins (FIGS. 3A-3D). Upon N and S protein addition, nanosensor ii (FIG. 3A) and nanosensor vi (FIG. 3B) show an instantaneous and continuous decrease in nIR signal on the order of 5 to 70% depending on protein concentrations. Nanosensor kinetic parameters were determined by fitting the maximum sensors response for 60 min to each analyte concentration series using the cooperative binding model of the Hill equation (ref. 35). For a first-order reversible reaction, the relationship between the analyte (A) and available docking sites (θ) for viral proteins can be described as follows:

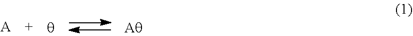

Where Aθ the surface concentration of analyte bound sensor sites. The resulting equilibrium for this reaction is:

$${K}_{A} = \frac{[ A θ ]}{[ A ] [ θ ]} \qquad ( 2 )$$

Assuming that the sensor response is proportional to the Aθ/θₜₒₜ ratio, it is found that

$$\frac{{I}_{0} - I}{{I}_{0}} = α \frac{[ A θ ]}{[ {θ}_{tot} ]} + β = α \frac{{( [ A ] {K}_{A} )}^{n}}{{( [ A ] {K}_{A} )}^{n} + 1} + β \qquad ( 3 )$$

with the total concentration of available recognition sites on the sensor a constant of [θ]ₜₒₜ and the parameter n for the analyte cooperativity. Fitting the data in FIGS. 3A-3B with equation (3) (R²=0.998 and 0.980) results in a proportionality factor α=61.91 and 0.683 with β=3.14 and −0.005, K_{D}=1/K_{A}=0.840 and 0.213 nM, and n=1.176 and 1.120 for N and S protein respectively, indicating positive cooperativity in good agreement with previous work (n>1) (FIGS. 3C-3D) (ref. 19). The K_{d} value of N and S protein nanosensors are lower than previously reported CoPhMoRe sensing which suggests influence of the larger molecular size of viral proteins, with more binding sites involved, and hence polyvalent interactions. The limit of detection in this mode is 49 fM and 350 pM for N and S protein respectively; this value was calculated by adding the nanosensor response from the addition of only buffer as the noise level to 3-times of the signal.

The SARS-CoV-2 nanosensor compatibility in biofluids was assessed by testing the response to viral proteins in 1% and 100% human saliva. For the N protein nanosensor, small sensor responses with 3.5 and 6.3% intensity change were observed with 1% and 100% saliva, respectively (left, FIG. 4A). However, significantly larger responses to N protein in 100% saliva were observed with 45.4% change demonstrating that N protein detection appears to be independent of background saliva effects. SARS-CoV-2 patients show N protein concentration range from 10 to 10⁴ pg/mL in their saliva during symptom of day 1 to 7 (ref. 36). Since detecting range of nanosensor vi (LOD of 2.4 pg/mL) sufficiently covers this clinical range, the CoPhMoRe nanosensor can diagnose the positive cases with real saliva sample. For the S protein nanosensor, on the other hand, the sensor responses to the analyte were clearly diminished in saliva conditions with almost similar changes for the control −S protein saliva and +S protein saliva samples (right, FIG. 4A). The attenuation of the highly glycosylated S protein nanosensor response seems to arise from the decreased baseline of the fluorescence with saliva background (refs. 37, 38). One hypothesis for future exploration is that the salivary glycoprotein can adsorb onto the S protein nanosensor surface and reduce the baseline fluorescence intensity and block viral protein binding. The N protein, on the other hand, is phosphorylated and the analyte-nanosensor interaction is not affected by glycoproteins present in saliva (ref. 39). The nIR fluorescence spectra demonstrate that the N protein nanosensor described herein shows almost identical responses in buffer and the 100% human saliva condition (FIG. 4B). This suggests that the PEG-phospholipid nanosensor passivation, built into this construct, enables at least partial reduction of nanosensor biofouling (ref. 21).

In order to demonstrate a form of the sensor compatible with diagnosis in a non-laboratory setting, the nanosensors in this work were interfaced into a Lab-on-Fiber system using a fiber optic-based benchtop instrument to produce sensor optode. All components including the optode fiber, laser, nanosensors, nIR detectors, monitor, controller and test solutions can be compactly integrated onto a mobile cart, for example (left, FIG. 4C). The optode fiber is flexible, lightweight, and robust enough such that the tip can be applied to samples for diagnosis with an ease not found with conventional analytical tools (ref. 40). 3D miniaturized sensor tips were designed and fabricated as interfaces that hold the SARS-CoV-2 nanosensor media and viral protein components stably onto the fiber optics using 3D-printing and a developed template (Objet 30 Prime, Stratasys Ltd). The sensing tip is engineered at high-resolution (mm-scale) in a specific 3D architecture such that viral proteins can be sensitively detected even with low volumes of biofluid extraction (currently <10 μL). The printed 3D sensing tip was successfully integrated with the optode fiber by screwing onto an SMA (SubMiniature version A) connector and supported by an 8 mm-cover glass slide for the Lab-on-Fiber SARS-CoV-2 diagnosis system (left, FIG. 4C). FIG. 4D shows the real-time fluorescence response of the completed, fully-integrated optode fiber to SARS-CoV-2 N protein in buffer (top graph) and in 100% human saliva condition (bottom graph). A 5 μL viral protein solution was directly dropped onto the 3D printed sensing tip of the optode fiber and the resulting sensor response was directly measured using the benchtop instrument. The optode fiber itself generates no response to pure buffer spiked with BSA, but the expected rapid and sizable turn-off response to N protein in buffer with a 71.3% change in intensity. For the 100% saliva condition, the optode fiber appears to show a background quenching response to pure saliva (labeled −SARS-CoV-2) with a 30% change, the origins of which are unclear at this time. However, saliva plus analyte (labeled +SARS-CoV-2) demonstrates a response of 85.7%, easily above the background and statistically significant. A response time (τ₉₀) of 5.1 min was achieved based on the time to reach 90% of the nIR level at infinity. Overall, the platform appears rapid enough for SARS-CoV-2 detection with small biofluid volume additions and short response time such that non-laboratory operation is possible.

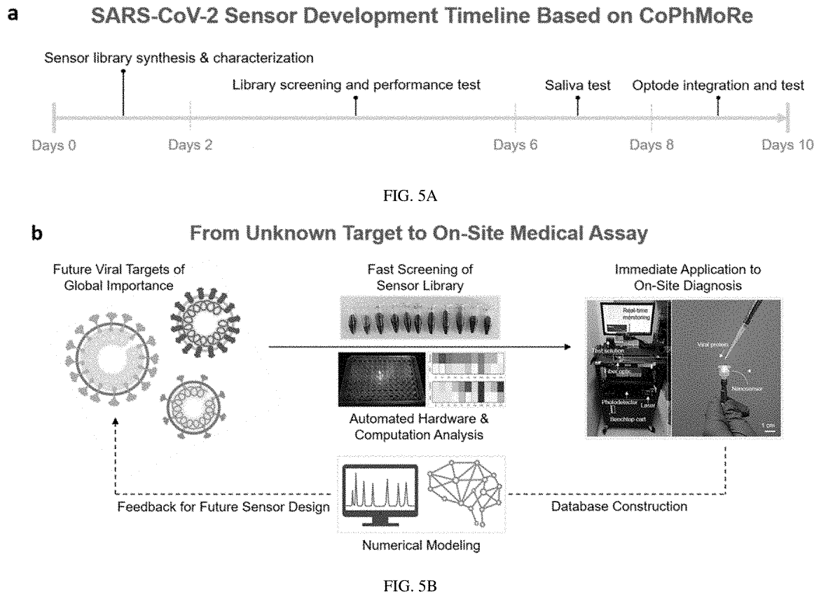

The development timeline and workflow necessary to generate a sensor for virial detection starting from identification of the new virial target itself to deployable hardware ready for screening of the population can lead to the generation of new sensors. The CoPhMoRe technique seems to allow for rapid development, with libraries that can be screened at an accelerated pace to find a sensor optimum for a given viral target. FIG. 5A shows the development timeline for this work following the experimental history of the project. The whole CoPhMoRe library was synthesized, performed characterization of the corona phases, screened against viral biomarkers and evaluated sensor performance for nanosensors for N and separately S protein targets within just 6 days of two researchers working 4 hours per day.

In addition, these optimized nanosensor candidates could be efficiently integrated into a modular optode fiber optic setup and tested against the relevant biofluid (saliva) within 4 days. This sensor development depends on the design and selection of proper corona phase. Thus, in cases where no commercial polymer or chemistry show satisfying sensitivity and/or selectivity towards the target analytes, the development timeline may be extended. A unique feature of this type of molecular recognition scheme is that rapid design and testing is possible, unhindered by the development time and supply chain requirements of a conventional antibody or enzymatic receptor. CoPhMoRe is also compatible with fully-automated fluorescence screening hardware that connects seamlessly to a benchtop optics platform. This rapid development workflow holds several potential advantages for future virial targets. Based on this development, the CoPhMoRe platform described herein can be readily applied to potential future pandemic with unknown viruses (FIG. 5B). If a new virus target begins to circulate, this workflow can generate a corona interface library and synthesize recognition candidates within a few days. Computational analysis can be used to predict selectivity and sensitivity performance in advance of experimental validation. Then, the optimized nanosensor with selective recognition for the unknown virial target can be interfaced directly to the existing bench top platform shown in FIG. 4C. Thus, the sensor produced from the CoPhMoRe-based sensor screening platform is potentially ready for field-deployment in advance of a future pandemic. In addition, based on the sensing and diagnosis data, a database of CoPhMoRe sensor library can be constructed onto a variety of viruses. In this way, the workflow allows for a potential feedback loop for continual sensor development targeting new, emerging viruses leveraging numerical modeling.

### Experimental Details

Materials

Raw single walled carbon nanotube (SWCNT) produced by HiPCO process were purchased from NanoIntegris and used without further processing (Batch #HR27-104). PEG-phospholipids were purchased from Avanti Polar Lipids Inc. All other chemicals were purchased from Sigma Millipore.

Nanosensor Synthesis and Characterizations

In 1 mL of DI water, 1 mg (1 equiv.) HiPCO SWCNT and 1 mg (1 equiv.) of PEG-phospholipid were mixed. The mixture was ultrasonicated using a ⅛″ probe (Cole-Parmer) at a power of 10 W for 30 minutes (QSonica). The resulting suspension was benchtop centrifuged twice at 30300 RCF for 1 hr (Eppendorf Centrifuge 5430R). The top 80% of the suspension was reserved for further use, while the remaining 20% was discarded. UV-vis-nIR absorption spectroscopy (Agilent Technologies, Cary 5000) was used to confirm successful suspensions and obtain the mass concentration of the nanoparticles using an extinction coefficient of ε₃₂=0.036 mg·L⁻¹·cm⁻¹ (ref. 41). The accessible surface area of the PEG-phospholipid wrapped SWCNT was measured using the molecular probe adsorption technique (ref. 32). Fluorescent emission (510 to 560 nm) intensity of riboflavin from to were measurement using a Thermo VarioSkan Plate, with excitation at 460 nm. Deflections of the riboflavin fluorescence were taken in the presence of nanosensor suspensions with SWCNT concentration of 10 mg/L.

nIR Signal Measurements

High throughput screening of the nanosensor library against the viral proteins was performed using a customized nIR microscope, which consists of a Zeiss Axio Vision inverted microscope body with a 20× objective, coupled to an Acton SP2500 spectrometer and liquid nitrogen cooled InGaAs 1D detector (Princeton Instruments). In a 96-well plate, one PEG-phospholipid/SWCNT sensor (0.5 mg/L) and one viral protein (N or S protein, 10 μg/mL) were mixed in a final volume of 200 μL in N protein buffer (15 mM Na2HPO4, 5 mM NaH2PO4, 0.25 M NaCl, pH 7.5) and S protein buffer (2 mM Tris, 200 mM NaCl, pH 8.0), and incubated for 1 hr in each well. The samples were then illuminated by a 150 mW 785 nm photodiode laser (B&W Tek Inc.), and fluorescence emission spectra were collected from 950 to 1250 nm. Peak position and intensities of each sensor-viral proteins pair were compared to a nanosensor/buffer and nanosensor/BSA control to calculate the selective sensor responses. The most promising candidates were identified and studied further. Proteins and saliva were sourced and reconstituted as listed in Table 1. 1% and 100% saliva are calculated from the saliva concentration of whole analyte solution including viral proteins.

TABLE 1
Proteins and saliva specifications used for the study.

| Purpose | Protein | Manufacturer | Item # | Source | Purification | Buffer | Concentration |
|---|---|---|---|---|---|---|---|
| Sensing | Spike-stable- | Department of | N/A | Expressed | Strep-Tactin | 2 mM Tris, | 1 mg/mL |
| protein | REXAPRO | Molecular | | in HEK- | affinity and size | 200 mM NaCl, | (~133.7 kDa) |
| | (S protein)* | Biosciences, | | 293 cells | exclusion | pH 8 | |
| | | University of | | | chromatography | | |
| | | Texas | | | | | |
| Sensing | Nucleocapsid | RayBio | 770-729- | Expressed | His-tag affinity | 15 mM | 1.45 mg/mL |
| protein | phosphoprotein | | 2992 | in Ecoli | purification by | Na₂HPO₄, | (~50 kDa) |
| | (Severe acute | | | (Expressed | immobilized | 5 mM | |
| | respiratory | | | region: | metal ion affinity | NaH₂PO₄, | |
| | syndrome | | | Metl - | chromatography | 0.25M NaCl, | |
| | coronavirus 2) | | | Ala419) | | pH 7.5 | |
| | GenBank: | | | | | | |
| | QHD43423.2 (N | | | | | | |
| | protein) | | | | | | |
| Control | Bovine Serum | Sigma Aldrich | A2153 | N14 | N/A | 2 mM Tris, | ~66 kDa |
| | Albumin | | | | | 200 mM NaCl, | |
| | (lyophilized | | | | | pH 8 | |
| | powder, ≥96% | | | | | 15 mM | |
| | (agarose gel | | | | | Na₂HPO₄, | |
| | electrophoresis)) | | | | | NaH₂PO₄, | |
| | | | | | | 0.25M NaCl, | |
| | | | | | | pH 7.5 | |
| Biofluid | Saliva | MyBioSource | MBSI70292 | Pooled | N/A | N/A | NA |
| | | | | Human | | | |
| | | | | Donors | | | |

*See Shsieh, C.-L., et al. Structure-Based Design of Prefusion-Stabilized SARS-CoV-2 Spikes. Science 2020, 369, 1501-1505, which is incorporated by reference in its entirety.

Optode Fiber Measurements

Sensors were excited with 561 nm (MGL-FN-561 200 mW, Opto Engine LLC) or 785 nm (MDL-Ill-785 500 mW, Opto Engine LLC). The laser light propagates through fiber optic reflection/backscatter probe bundles (RP29, Thorlabs) to the samples, and the fluorescence light propagates through the fiber to InGaAs amplified photodetector (PDF10C, Thorlabs). The fiber optic probe consists of 6 fibers around 1 fiber configuration where the central fiber provides the light delivery to the PEG-phospholipid/SWCNT nanosensor. The surrounding 6 fibers collect the near infrared fluorescence light from nanosensor. To reduce laser scattering and autofluorescence at hydrogel, 900 nm short pass filter and 900 nm long pass filter were inserted at the laser and photodetector, respectively. A focusing lens with a focal length of 30 mm is placed to efficiently collect fluorescence signal at 0.5 mm-diameter active area of the photodetector. All the components of the instrument are loaded on a mobile cart (15Y320, Grainger, size: depth 18″, width 24″, height 26-42″). For the real-time signal measurement of SARS-CoV-2, 5 μL buffer, BSA (10 μg/mL), and N protein (10 μg/mL) were added in series and fluorescence signals were measured for 30 min. 3D sensing tips were fabricated using a 3D printer (Objet 30 Prime, Stratasys Ltd.). The print was executed using “High Quality” settings and VeroClear (PN: OBJ-04055, Stratasys) material with a minimum layer thickness of 16 μm. The prints were washed from the support material using an electric power washer (supplied by Stratasys). Resolution of 3D printer is X-axis: 600 dpi; Y-axis: 600 dpi; Z-axis: 1600 dpi and accuracy is 0.1 mm (0.0039″).

The molecular probe adsorption (MPA) technique was used to measure the accessible surface area of the polymer-wrapped SWCNT using riboflavin. See, for example, Park, M., et al. Nano Lett. 2019, 19, 7712-7724, which is incorporated by reference in its entirety. A type 1 Langmuir isotherm was used to describe the probe adsorption process. Assume a free probe concentration of C_{probe}, the concentration of the SWNT adsorbed probe is C_{probe,SWCNT}, the concentration of the polymer adsorbed probe is C_{probe,pol}, and the total concentration of probe is Cₜₒₜₐₗ. The total number of vacant sites on polymer wrapped SWCNT is q (per carbon atom), the total concentration of vacant sites on SWCNT is θₜ, the concentration of probe adsorbed sites on SWCNT is θₐ, the concentration of free sites after probe adsorption is θ, the forward reaction rate is k_{forward}, the backward reaction rate is k_{backward}, the dissociation constant of probe binding to SWCNT is K_{D}, and the SWCNT concentration on a carbon atom basis is C_{SWCNT}.

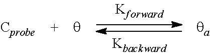

At equilibrium, the forward and backward reactions have the same rate:

$${C}_{probe} \cdot θ \cdot {k}_{forward} = {θ}_{a} \cdot {k}_{backward} and$$

$${K}_{D} = \frac{{k}_{backward}}{{k}_{forward}} and$$

$${θ}_{t} = {C}_{SWNT} \cdot q$$

Therefore, the above equation can be converted to:

$${C}_{probe} ( {θ}_{t} - {θ}_{a} ) = {K}_{D} {θ}_{a}$$

$${C}_{probe , SWNT} = {θ}_{a} = \frac{{C}_{probe} {θ}_{t}}{{K}_{D} + {C}_{probe}} = \frac{{C}_{probe} \cdot {C}_{SWNT} \cdot q}{{K}_{D} + {C}_{probe}}$$

Similarly, the amount of probe adsorbed to the free polymer is:

$${C}_{probe , pol} = \frac{{C}_{probe} \cdot {C}_{pol} \cdot {q}_{pol}}{{K}_{D , pol} + {C}_{probe}}$$

The total riboflavin concentration in the solution is:

$${C}_{tot} = {C}_{probe} + \frac{{C}_{probe} \cdot {C}_{pol} \cdot {q}_{pol}}{{K}_{D , pol} + {C}_{probe}} + \frac{{C}_{probe} \cdot {C}_{SWNT} \cdot q}{{K}_{D} + {C}_{probe}}$$

Assuming K_{D,pol} is an order of magnitude higher than C_{probe}:

$${qC}_{SWNT} \frac{{C}_{probe}}{{K}_{D} + {C}_{probe}} = {C}_{total} - {C}_{probe} - \frac{{q}_{pol} {C}_{pol}}{{K}_{D , pol}} {C}_{probe} = Δ$$

Inverting the above equation, one finds:

$$\frac{{C}_{SWNT}}{Δ} = \frac{{K}_{D}}{q} \frac{1}{{C}_{probe}} + \frac{1}{q}$$

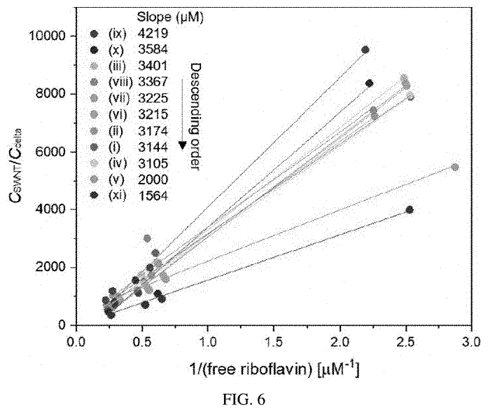

The resulting linear regression (FIG. 6) between

$$\frac{{C}_{SWNT}}{Δ} and \frac{1}{{C}_{probe}}$$

are used to determine the parameter

$$\frac{q}{{K}_{D}}$$

(inverse of the slope).

Reference numbers in parenthesis “(ref.-)” herein refer to the corresponding literature listed in the attached Bibliography, which forms a part of this Specification, and each of the referenced literature documents in the followed list is incorporated by reference in its entirety.
[1] Kronbichler, A.; et al. Asymptomatic Patients as a Source of COVID-19 Infections: A Systematic Review and Meta-Analysis. Int. J. Infect. Dis. 2020, 98, 180-186.
[2] Udugama, B.; et al. Diagnosing COVID-19: The Disease and Tools for Detection. ACS Nano 2020, 14, 3822-3835.
[3] Pokhrel, P.; et al. Detecting the Coronavirus (COVID-19). ACS Sens. 2020, 5, 2283-2296.
[4] Esbin, M. N.; et al. Overcoming the Bottleneck to Widespread Testing: A Rapid Review of Nucleic Acid Testing Approaches for COVID-19 Detection. RNA 2020, 26, 771-783.
[5] Mio, C.; et al. A Streamlined Approach to Rapidly Detect SARS-CoV-2 Infection Avoiding RNA Extraction: Workflow Validation. Dis. Markers 2020, 2020, 8869424.
[6] McMinn, S.; et al. In Large Texas Cities, Access To Coronavirus Testing May Depend On Where You Live. at www.npr.org/sections/health-shots/2020/05/27/862215848/across-texas-black-and-hispanic-neighborhoods-havefewer-coronavirus-testing-sit (accessed Jun. 17, 2021).
[7] Kim, S.; et al. Vertical Flow Cellulose-Based Assays for SARS-CoV-2 Antibody Detection in Human Serum. ACS Sens. 2021, 6, 1891-1898.
[8] EUA Authorized Serology Test Performance at www.fda.gov/medical-devices/coronavirus-disease-2019-covid-19-emergency-use-authorizations-medical-devices/eua-authorized-serology-test-performance (Accessed on Jun. 17, 2021).
[9] Whitman, J. D.; et al. Test Performance Evaluation of SARS-CoV-2 Serological Assays. medRxiv Preprint 2020, DOI: 10.1101/2020.04.25.20074856.
[10] Vandenberg, O.; et al. Considerations for Diagnostic COVID-19 Tests. Nat. Rev. Microbiol. 2021, 19, 171-183.
[11] American Society for Microbiology. Laboratory Supply Shortages Are Impacting COVID-19 and Non-COVID Diagnostic Testing at asm.org/Articles/2020/September/Laboratory-Supply-Shortages-Are-Impacting-COVID-19 (accessed on Jun. 18, 2021).
[12] Seo, G.; et al. Rapid Detection of COVID-19 Causative Virus (SARS-CoV-2) in Human Nasopharyngeal Swab Specimens Using Field-Effect Transistor-Based Biosensor. ACS Nano 2020, 14, 5135-5142.
[13] Shao, W.; et al. Rapid Detection of SARS-CoV-2 Antigens Using High-Purity Semiconducting Single-Walled Carbon Nanotube-Based Field-Effect Transistors. ACS Appl. Mater. Interfaces 2021, 13, 10321-10327.
[14] Zhang, X.; et al. Electrical Probing of COVID-19 Spike Protein Receptor Binding Domain via A Graphene Field-Effect Transistor. arXiv:2003.12529; at arxiv.org/abs/2003.12529 (accessed Jun. 17, 2021).
[15] Guo, K.; et al. Rapid Single-Molecule Detection of COVID-19 and MERS Antigens via Nanobody-Functionalized Organic Electrochemical Transistors. Nat. Biomed. Eng. 2021, doi.org/10.1038/s41551-021-00734-9.
[16] Mahari, S.; et al. eCovSens-Ultrasensitive Novel In-House Built Printed Circuit Board Based Electrochemical Device for Rapid Detection of nCovid-19 antigen, a spike protein domain 1 of SARS-CoV-2. bioRxiv Preprint 2020, DOI: 10.1101/2020.04.24.059204.
[17] Beduk, T.; et al. Rapid Point-of-Care COVID-19 Diagnosis with a Gold-Nanoarchitecture-Assisted Laser-Scribed Graphene Biosensor. Anal. Chem. 2021, 93, 8585-8594.
[18] Qiu, G.; et al. Dual-Functional Plasmonic Photothermal Biosensors for Highly Accurate Severe Acute Respiratory Syndrome Coronavirus 2 Detection. ACS Nano 2020, 14, 5268-5277.
[19] Ahmadivand, A.; et al. Functionalized Terahertz Plasmonic Metasensors: Femtomolar-Level Detection of SARS-CoV-2 Spike Proteins. Biosens. Bioelectron. 2021, 177, 112971.
[20] Chen, H.; et al. Sensitive Detection of SARS-CoV-2 Using a SERS-Based Aptasensor. ACS Sens. 2021, 6, 2378-2385.
[21] Pinals, R. L.; et al. Rapid SARS-CoV-2 Spike Protein Detection by Carbon Nanotube-Based Near-Infrared Nanosensors. Nano Lett. 2021, 21, 2272-2280.
[22] Chen, R.; et al. Early Detection of SARS-CoV-2 Seroconversion in Humans with Aggregation-Induced Near-Infrared Emission Nanoparticle-Labeled Lateral Flow Immunoassay. ACS Nano 2021, 15, 8996-9004.
[23] Moitra, P.; et al. Selective Naked-Eye Detection of SARS-CoV-2 Mediated by N Gene Targeted Antisense Oligonucleotide Capped Plasmonic Nanoparticles. ACS Nano 2020, 14, 7617-7627.
[24] Shan, B.; et al. Multiplexed Nanomaterial-Based Sensor Array for Detection of COVID-19 in Exhaled Breath. ACS Nano 2020, 14, 12125-12132.
[25] Du, L.; et al. The Spike Protein of SARS-CoV—a Target for Vaccine and Therapeutic Development. Nat. Rev. Microbiol. 2009, 7, 226-236.
[26] Lan, J.; et al. Structure of the SARS-CoV-2 Spike Receptor-Binding Domain Bound to the ACE2 Receptor. Nature 2020, 581, 215-220.
[27] Jia, H.; et al. A graphene oxide coated tapered microfiber acting as a super-sensor for rapid detection of SARS-CoV-2. Lab Chip. 2021, 21, 2398-2406.
[28] Saerens, D.; et al. Antibody Fragments as Probe in Biosensor Development. Sensors 2008, 8, 4669-4686.
[29] Peluso, P.; et al. Optimizing Antibody Immobilization Strategies for the Construction of Protein Microarrays. Anal. Biochem. 2003, 312, 113-124.
[30] Charter, N. W.; et al. Rapid, Antibody-Free Detection of Recombinant Proteins on Blots Using Enzyme Fragment Complementation. Methods Mol. Biol. 2009, 536, 395-405.
[31] Bisker, G.; et al. Protein-Targeted Corona Phase Molecular Recognition. Nat. Commun. 2016, 7, 10241.
[32] Park, M.; et al. Measuring the Accessible Surface Area within the Nanoparticle Corona Using Molecular Probe Adsorption. Nano Lett. 2019, 19, 7712-7724.
[33] Zhang, J. Q.; et al. Molecular Recognition Using Corona Phase Complexes Made of Synthetic Polymers Adsorbed on Carbon Nanotubes. Nat. Nanotechnol. 2013, 8, 959-968.
[34] Lee, M. A.; et al. Implantable Nanosensors for Human Steroid Hormone Sensing In Vivo Using a Self-Templating Corona Phase Molecular Recognition. Adv. Healthcare Mater. 2020, 9, 2000429.
[35] Kruss, S.; et al. Neurotransmitter Detection Using Corona Phase Molecular Recognition on Fluorescent Single-Walled Carbon Nanotube Sensors. J. Am. Chem. Soc. 2014, 136, 713-724.
[36] Shan, D.; et al. N-Protein Presents Early in Blood, Dried Blood and Saliva During Asymptomatic and Symptomatic SARS-CoV-2 Infection. Nat. Commun. 2021, 12, 1931.
[37] Herndández, E. R.; et al. The Role of the SARS-CoV-2 S-Protein Glycosylation in the Interaction of SARS-CoV-2/ACE2 and Immunological Responses. Viral Immunol. 2021, 34, 165-173.
[38] Watanabe, Y.; et al. Site-Specific Glycan Analysis of the SARS-CoV-2 Spike. Science 2020, 369, 330-333.
[39] Bouhaddou, M.; et al. The Global Phosphorylation Landscape of SARS-CoV-2 Infection. Cell 2020, 182, 685-712.
[40] Kozawa, D.; et al. A Fiber Optic Interface Coupled to Nanosensors: Applications to Protein Aggregation and Organic Molecule Quantification. ACS Nano 2020, 14, 10141-10152.
[41] Zhang, J.; et al. A Rapid, Direct, Quantitative, and Label-Free Detector of Cardiac Biomarker Troponin T Using Near-Infrared Fluorescent Single-Walled Carbon Nanotube Sensors. Adv. Healthc. Mater. 2014, 3, 412-423.

It should be understood that the subject matter defined in the appended claims is not necessarily limited to the specific implementations described above. The specific implementations described above are disclosed as examples only.

What is claimed is:

1. A sensor comprising a nanoparticle structure including a lipid functionalized polyethylene glycol associated with a carbon nanotube.

2. The sensor of claim 1, wherein the lipid functionalized polyethylene glycol is a phospholipid functionalized polyethylene glycol.

3. The sensor of claim 1, wherein the lipid functionalized polyethylene glycol is a polyethylene glycol-phospholipid heteropolymer.

4. The sensor of claim 1, wherein the lipid functionalized polyethylene glycol has the formula:
(R₁ [symbol] ₙL-Polymer
wherein:
Polymer is a polyethylene glycol;
L is a linking group including an ester, an ether, a phosphate, a thioester, a sulfide, a sulfoxide, a sulfate, a phosphonate, a carbonate, a carbamate, or a carbamide group;
n is 1, 2, or 3; and
each R₁ is, independently, a C6-C24 alkyl chain.

5. The sensor of claim 1, wherein the polyethylene glycol has a terminal group selected from an ether, an ester, a carboxylic acid, or an amine.

6. The sensor of claim 1, wherein the polyethylene glycol has an average molecular weight of between 200 and 10,000.

7. The sensor of claim 1, wherein the polyethylene glycol has an average molecular weight of about 500, 1000, 2000, 3000, 4000, or 5000.

8. The sensor of claim 1, wherein each Rₜ is, independently, a C8, C10, C12, C14, C16, C18, C20, C22, or C24 alkyl chain.

9. The sensor of claim 1, wherein the lipid functionalized polyethylene glycol is selected from the group consisting of compounds of structures (i) to (xi):

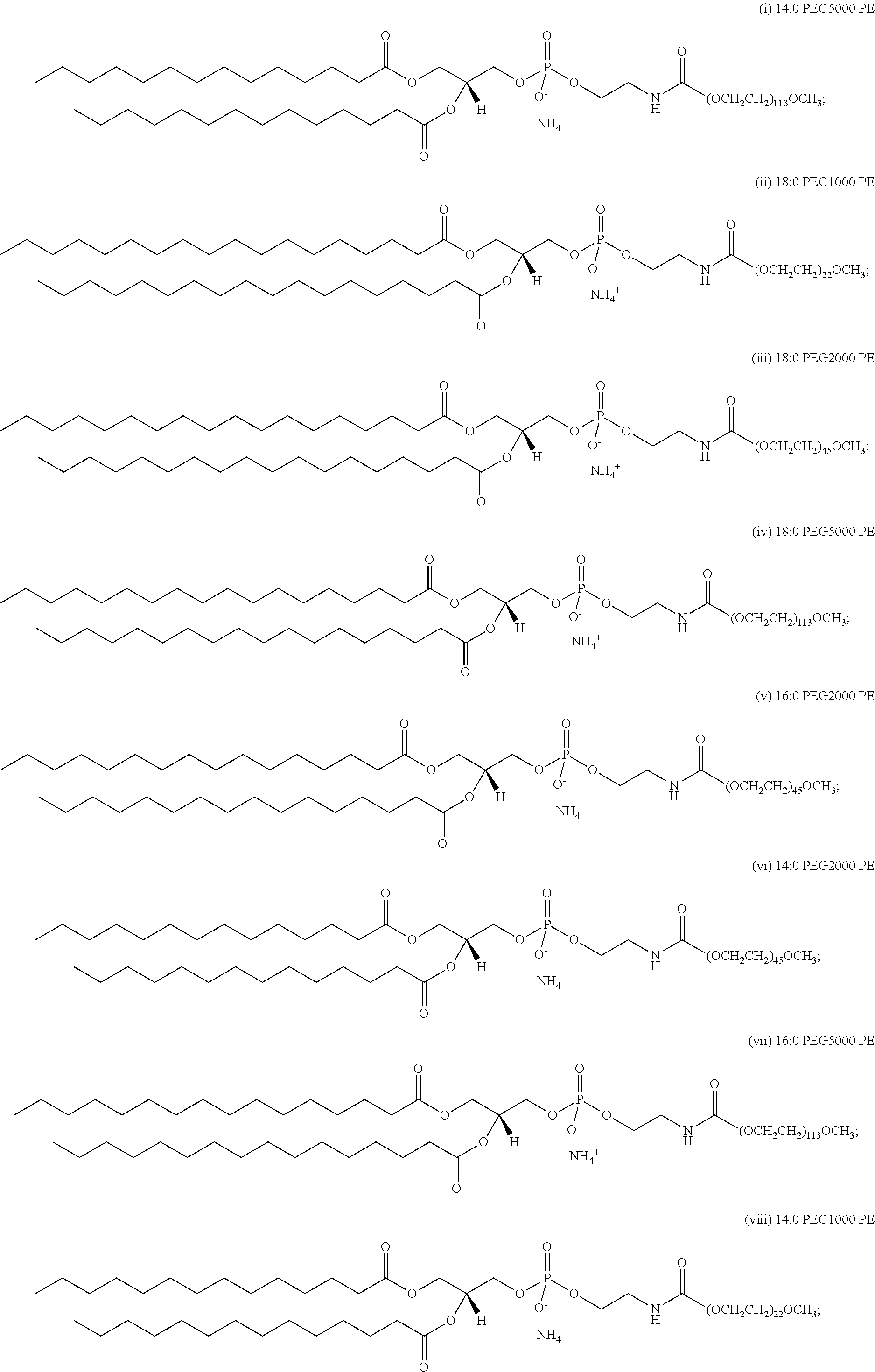

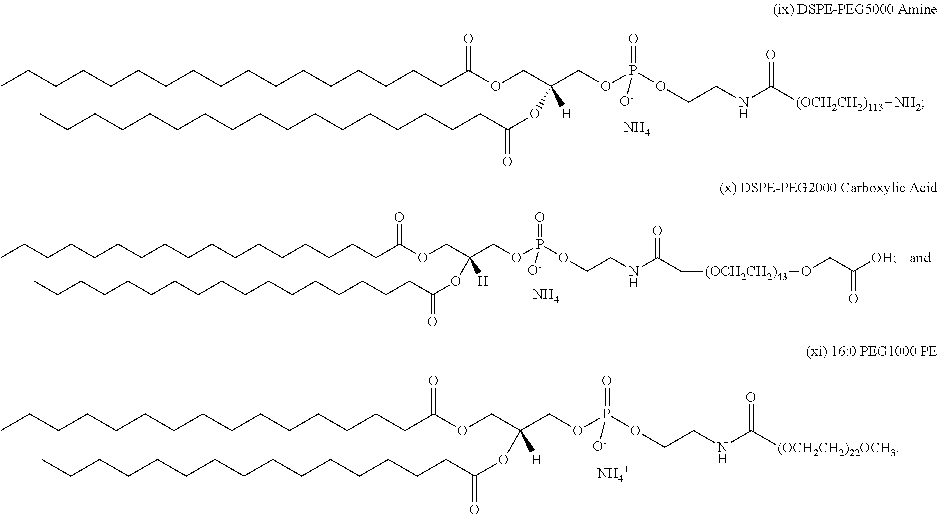

10. The sensor of claim 1, wherein the carbon nanotube is a single-walled carbon nanotube.

11. The sensor of claim 10, wherein the single-walled carbon nanotube has a diameter of about 0.8 to 1.2 nm.

12. The sensor of claim 1, wherein the carbon nanotube has a near infrared fluorescence that modulates in the presence of an analyte.

13. The sensor of claim 12, wherein the analyte includes a nucleocapsid (N) protein or a spike (S) protein of a coronavirus, or a combination thereof.

14. The sensor of claim 13, wherein the coronavirus comprises a SARS-CoV-2 type virus.

15. The sensor of claim 13, wherein the sensor is configured to detect the S protein and the lipid functionalized polyethylene glycol comprises the structure of compound (vi)

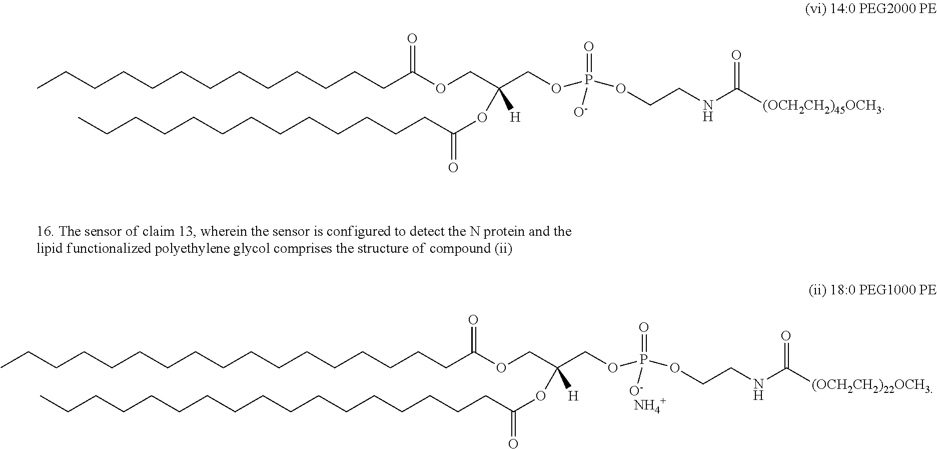

36. The sensor of claim 13, wherein the sensor is configured to detect the N protein and the lipid functionalized polyethylene glycol comprises the structure of compound (ii)

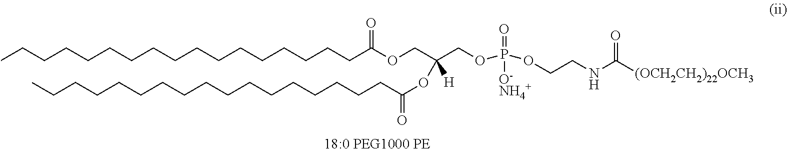

17. The sensor of claim 1, further comprising an excitation source and an emission detector.

18. The sensor of claim 17, further comprising a three-dimensional sensing tip with an optical connection for the excitation source and the emission detector.

19. The sensor of claim 17, further comprising a microscope system including the excitation source and the emission detector.

20. A method of detecting presence of a coronavirus in a sample comprising:
contacting the sample with a sensor of claim 1; and
measuring an amount of near infrared fluorescence emitted from the sensor.

21. The method of claim 20, wherein the sample includes saliva.

22. The method of claim 20, wherein the amount of near infrared fluorescence emitted detector detects the N protein or the S protein, or both, of the coronavirus at a limit of detection at a concentration between about 40 fM and 400 pM.

23. A method of developing a sensor for a virus comprising:
selecting a polymer screening array based on one or more structural features of the virus;
assembling a plurality of test sensors by associating each member of the polymer screening array with a carbon nanotube;
assessing a response of each of the plurality of test sensors to the one or more structural features of the virus; and
selecting the sensor based on the response.
